(19) United States
(12) Patent Application Publication
(10) Pub. No.: US 2023/0220497 A1
(43) Pub. Date: Jul. 13, 2023

(54) NUCLEIC ACID AMPLIFICATION ASSAY USING 3-D MAGNETIC RESONANCE IMAGING DETECTION FOR SCREENING LARGE POPULATIONS

(71) Applicant: Loganathan Doraisamy, San Diego, CA (US)

(72) Inventor: Loganathan Doraisamy, San Diego, CA (US)

(21) Appl. No.: 17/925,308

(22) PCT Filed: May 16, 2021
(86) PCT No.: PCT/US2021/032650

§ 371 Date: Nov. 14, 2022

Related U.S. Application Data
(60) Provisional application No. 63/133,431, filed on Jan. 4, 2021.
(60) Provisional application No. 62/704,540, filed on May 14, 2020.

(51) Int. Cl. C12Q 1/70; C12Q 1/6853; G01N 24/08

(52) U.S. Cl. CPC C12Q 1/701; C12Q 1/6853; G01N 24/088; C12Q 1/6806

(57) ABSTRACT

The present invention provides methods for high throughput screening and detection of nucleic acids from pathogens, such as SARS CoV-2, using nucleic acid amplification with nanoparticle binding complex formation and MRI or NMR detection. In certain embodiments, the MRI is three-dimensional MRI that simultaneously detects a plurality of amplified nucleic acid-nanoparticle complexes. In certain embodiments, the nucleic acids are amplified by isothermal LAMP techniques. In other embodiments, the nucleic acids are amplified by PCR. Methods of the invention are particularly useful rapid screening of large number of samples during pandemic situations.

### RELATED APPLICATIONS

This application claims the benefit of priority under 35 USC § 119 of U.S. Provisional Patent Applications Ser. No. 62/704,540 (filed May 14, 2020), and 63/133,431 (filed Jan. 4, 2021), the entire disclosures of which are incorporate herein by reference.

### FIELD OF THE INVENTION

The present invention relates to methods and systems for simultaneously detecting the presence or absence of one or more target nucleic acid sequences in large numbers of samples, using three-dimensional imaging. Specifically, the present invention relates to large-scale, sensitive, and rapid detection of nucleic acid sequences in human clinical specimens using three dimensional (3-D) magnetic resonance detection. The methods of the invention are particularly useful in diagnostic screening for the presence of microbiological pathogens (e.g., viruses, bacteria, fungi) in biological specimens (e.g., nasal swabs, saliva, sputum, blood and the like) from large populations of humans and/or non-human animals, as may be needed during endemic, epidemic, and particularly pandemic disease situations.

### BACKGROUND

Detecting the presence of particular nucleic acid sequences using nucleic acid amplification methods is a powerful diagnostic tool. Polymerase chain reaction (PCR), Loop Mediated Isothermal Amplification (LAMP) and other methods have allowed rapid amplification of specific target DNA sequences present in a multitude of samples. The discovery and clinical exploitation of reverse transcription (RT) amplification has made possible detection of genetic material from RNA viruses (e.g., influenza, SARS, MERS, COVID-19, Dengue Virus, hepatitis C, hepatitis E, West Nile fever, Ebola virus, rabies, polio, mumps, and measles viruses) as well as DNA viruses (e.g., papillomaviruses, pox viruses, herpesviruses, and adenoviruses), bacteria, fungi, plants and animals, including human beings, and life forms.

The recent SARS-CoV-2 pandemic has demonstrated the urgent need and importance of rapid, high throughput, large-scale, surveillance of viral pathogens. The rapid spread of SARS-CoV-2 virus and related coronaviruses (causing severe acute respiratory syndrome (SARS) and Middle East respiratory syndrome (MERS)), has proven to be very dangerous and resulted in large parts of the world shutting down for an extended time to stop viral transmission. Efforts to control the spread of such deadly viruses requires rapid detection, so that infected individuals can be identified, tracked and isolated.

COVID-19 is a respiratory tract infection caused by a newly emergent coronavirus—Severe Acute Respiratory Syndrome coronavirus 2 (SARS-CoV-2)—which was first recognized in Wuhan, Hubei Province, China, in December 2019. Sequencing of the virus suggested that SARS-CoV-2 is a beta corona virus closely linked to SARS coronavirus 1. See Wu et al., “A new coronavirus associated with human respiratory disease in China” Nature 579:265 (2020).

Screening and diagnosing hundreds of millions of people infected with SARS-CoV-2 globally requires methods and systems that could process hundreds of thousands of clinical samples per day at low cost. Where such systems were slow to achieve these goals, infections skyrocketed and many people died from COVI-19.

The standard molecular diagnostic test for RNA viruses such as SARS-CoV-2, is a multistep process involving viral RNA extraction and quantitative reverse transcriptase PCR (RT-qPCR). Although many companies have produced RT-PCR kits to amplify the viral RNA, RNA extraction at any scale, is challenging. In a diagnostic laboratory it is performed on a limited number of samples at a time, using automated platforms that require specific reagents and consumables. This inflexibility has led to significant effort to build large new laboratories with existing research equipment to increase testing capacity, and to extract RNA on more open platforms that enable non-specific reagents and plastics to be used.

Large scale, rapid screening of human samples such as nasal swab and oral saliva samples collected from millions of people living urban centers within a short period of time would undoubtedly help prevent the spread of the pandemics. Large scale screening requires multiple steps that require coordinated efforts and systems to automate the process. These steps include:
a. sample collection,
b. sample packaging and transportation to processing locations,
c. virus inactivation and sample lysis to kill the pathogen and releases genetic material,
d. nucleic acid purification and concentration,
e. reverse transcription for viral RNA,
f. nucleic acid amplification using LAMP or PCR,
g. detection of amplified DNA,
h. visualization of the results via data processing
i. identification of positive and negative samples in the collected samples and translation to diagnosis of individuals,
j. optional—identification of the virus strain and variants thereof.

These steps must be properly organized to obtain rapid, reliable, accurate and actionable results from sample to answer in a timely and cost-effective manner and thereby intercede to stop the disease spread via isolation, treatment and contact tracing.

In the current COVID-19 pandemic, the prior art method of traditional RT-PCR provided limited capacity for screening only small numbers of human samples per day per system. In traditional disease surveillance, there was no need for large scale screening, but that limitation proved fatal to many victims of COVID-19. With the available technologies, screening large populations took more than one day and up to more than a week in the beginning of the pandemic. With the available screening methods, by the time infected individuals were identified, the virus had spread to even more people, making this pandemic uncontrolled as it spread throughout the world.

Several bottlenecks have been identified, which did not allow testing capacity to meet the demand and need, including: nucleic acid extraction from human clinical samples required large number of steps that needed complex automation equipment, large amount of consumables and large numbers of scientifically trained technicians to perform the extraction; large number of healthcare workers were needed to collect nasal samples from people, with no adequate means for self-collection; expensive, sophisticated real-time PCR and real-time LAMP instruments, large supply of reagents, large amount of personal protective equipment and a large amount of lab space was needed to process hundreds of millions of human samples every day, all over the world.

The use of PCR methodology significantly slowed the process as specifically designed instruments were needed to rapidly vary the temperature of the samples every minute or during tens of amplifications cycle, and such instruments have limited sample capacity, particularly when real-time PCR is used which uses and even more sophisticated and expensive instrument that has dedicated detector for every sample, each of which tracks the sample quantity in real time plots the results graphically. A simpler method, such as LAMP, performs nucleic acid amplification at a single temperature and might overcome this bottleneck.

In light of the experience with COVID-19, there is a continuing need for pathogen detection methods that are rapid, high throughput and nimble enough to be rapidly adapted and deployed when new pathogens emerge.

### SUMMARY OF THE INVENTION

The present invention provides methods for detecting a target nucleic acid comprising the steps of: a) providing a sample containing the target nucleic acid; b) amplifying the target nucleic acid with at least one primer, wherein the at least one primer is biotinylated, thereby preparing a biotinylated target nucleic acid; c) reacting the biotinylated target nucleic acid with streptavidin-nanoparticles, thereby forming amplified target nucleic acid-nanoparticle complexes; and d) detecting the nucleic acid-nanoparticle complexes with MRI or NMR. In certain embodiments, amplifying the target nucleic acid comprises LAMP or PCR. RT-LAMP or RT-PCR, and can comprises reverse transcription.

In certain embodiments, the sample is a human clinical sample such as saliva or a nasal swab.

In certain aspects, the sample is treated with at least one of: a chelating agent, proteinase K, guanidium hydrochloride, guanidium compositions and combinations thereof lyse or dissociate cells or release the nucleic acid from associated proteins, but typically does not require isolation or purification of the nucleic acid.

The target nucleic acid can be a nucleic acid of a pathogen, such as a pathogenic virus or bacteria, such as SARS-CoV-2 or a variant thereof.

The present invention also provides methods for screening a plurality of samples as described above, for the presence of a target nucleic acid comprising the steps of:

a) providing a plurality of clinical samples containing the target nucleic acid;
b) amplifying the target nucleic acid in each of the plurality of clinical samples with at least one primer, wherein the at least one primer is biotinylated, thereby preparing a plurality of biotinylated target nucleic acids samples;
c) reacting each of the plurality of biotinylated target nucleic acid samples with streptavidin-conjugated nanoparticles, thereby forming a plurality of amplified target nucleic acid-nanoparticle complexes; and
d) detecting the plurality of nucleic acid-nanoparticle complexes with MRI or NMR.

Amplifying the target nucleic acid can comprises LAMP or PCR or RT-LAMP or RT-PCR. in certain aspects, the amplifying the target nucleic acid comprises reverse transcription.

In specific embodiments the MRI is 3-D MRI, which simultaneously detects the plurality of nucleic acid-nanoparticle complexes.

### BRIEF DESCRIPTION OF THE DRAWINGS

FIG. 1. is a representative MRI image obtained from LAMP reaction for positive and negative samples are shown in an embodiment of the invention.

FIG. 2 is a representative MRI image obtained from LAMP reaction for positive and negative samples are shown in another second embodiment of the invention.

FIG. 3 is an image illustrating how the magnetic nanoparticles bind to un-used primers after a LAMP reaction, forming strong association between nanobeads, which is an embodiment resulting in FIG. 1 MRI image.

FIG. 4 is an MRI image illustrating MRI detected PCR and RT-PCR detection.

FIG. 5 illustrates a process for detecting a DNA analyte according to an embodiment of the invention. Panel A illustrates the template, primers and nanoparticles used. Panel B shows illustrates the method steps in the process. B=biotin; S=Streptavidin; NP=Nanoparticle core.

FIG. 6A illustrates the random orientation of the spin of non-zero spin, NMR-active nuclei.

FIG. 6B shows the effect of a uniform magnetic field on the NMR-active nuclei of FIG. 6A. FIG. 6C shows the effect of a radiofrequency RF pulse on the oriented NMR-active nuclei of FIG. 6B

FIG. 7 illustrates spin-lattice relaxation (T₁) (top panel) and spin-spin relaxation (T₂) (bottom panel) for an NMR-active nucleus. In the A panels, the nucleus is shown in a uniform magnetic field, oriented in the direction of the bulk magnetization vector. The nucleus reorients in response to a radiofrequency pulse (B panels) and will eventually “relax” toward the orientation of the bulk magnetization vector (C panels).

### DETAILED DESCRIPTION

### Definitions

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. As used herein, the terms “comprises,” “comprising”, “includes”, and “including”, or any other variations thereof, are intended to cover a non-exclusive inclusion, such that a process, method, composition, reaction mixture, kit, or apparatus that comprises a list of elements does not include only those elements but may include other elements not expressly listed or inherent to such process, method, composition, reaction mixture, kit, or apparatus. The section headings used herein are for organizational purposes only and are not to be construed as limiting the subject matter described.

Unless specific definitions are provided, the nomenclatures utilized in connection with, and the laboratory procedures and techniques of molecular biology, biochemistry, and organic chemistry described herein are those known in the art. Standard chemical and biological symbols and abbreviations are used interchangeably with the full names represented by such symbols and abbreviations. Thus, for example, the terms “deoxyribonucleic acid” and “DNA” are understood to have identical meaning. Standard techniques may be used e.g., for chemical syntheses, chemical analyses, recombinant DNA methodology, and oligonucleotide synthesis. Reactions and purification techniques may be performed e.g., using kits according to manufacturer's specifications, as commonly accomplished in the art or as described herein. The foregoing techniques and procedures may be generally performed according to conventional methods well known in the art and as described in various general or more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)); Maniatis et al., 1982 Molecular Cloning, Cold Spring Harbor Laboratory, Plainview, N.Y.; Harlow & Lane, Antibodies: A Laboratory Manual (Cold Spring Harbor Laboratory Press, Cold Spring Harbor, N.Y. (1988)); Rieger et al., 1991 Glossary of genetics: classical and molecular, 5th Ed., Berlin: Springer-Verlag; Current Protocols in Molecular Biology, F. M. Ausubel et al., Eds., Current Protocols, a joint venture between Greene Publishing Associates, Inc. and John Wiley & Sons, Inc., (1998 Supplement); Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York, which are incorporated herein by reference in their entirety for any purpose.

Definitions of common terms in chemistry may be found in: Compendium of Chemical Terminology (IUPAC recommendations), 2^{nd} Ed., 1997, Blackwell Science Inc, Oxford, U.K. (McNaught, ed.), which is available in a searchable online form on the world wide web at goldbook (dot) iupac (dot) org., which is incorporated herein by reference in its entirety for any purpose. Principles, methods, calculations and terminology related to NMR can be found in: Levitt, 2008, Spin Dynamics: Basics of Nuclear Magnetic Resonance John, 2^{nd} Ed., John Wiley & Sons, Inc., West Sussex, England; Palmer, III, et al., 2005, Protein NMR Spectroscopy, Second Edition: Principles and Practice, Elsevier Academic Press, San Diego, Calif.; Ernst et al. (eds.), 1990, Principles of Nuclear Magnetic Resonance in One and Two Dimensions, Oxford University Press, Oxford, UK; Mehring et al., 2001, Object-Oriented Magnetic Resonance: Classes and Objects, Calculations and Computations, Academic Press, San Diego, Calif.; Abragam, 1961, The Principles of Nuclear Magnetism, (International Series of Monographs on Physics, Book 32), Oxford: Clarendon Press, Clarendon, UK; Slichter, 1990, Principles of Magnetic Resonance, Springer, Berlin; Fukushima et al., 1981, Experimental Pulse NMR: A Nuts and Bolts Approach, Addison-Wesley, London; which are incorporated herein by reference in their entirety for any purpose.

It is to be understood that as used in the specification and in the claims, “a” or “an” can mean one or more, depending upon the context in which it is used. Thus, for example, reference to “a cell” can mean that at least one cell, two cells, or a plurality (three or more) of cells.

“About” as used herein means that a number referred to as “about” comprises the recited number plus or minus 1-10% of that recited number. For example, “about” 50 nucleotides can mean 45-55 nucleotides or as few as 49-51 nucleotides depending on the situation. Whenever it appears herein, a numerical range, such as “45-55”, refers to each integer in the given range; e.g., “45-55 nucleotides” means that the nucleic acid can contain 45 nucleotides, 46 nucleotides, etc., up to and including 55 nucleotides. Where a range described herein includes decimal values, such as “1.2% to 10.5%”, the range refers to each decimal value of the smallest increment indicated in the given range; e.g. “1.2% to 10.5%” means that the percentage can be 1.2%, 1.3%, 1.4%, 1.5%, etc. up to and including 10.5%; while “1.20% to 10.50%” means that the percentage can be 1.20%, 1.21%, 1.22%, 1.23%, etc. up to and including 10.50%.

“Transcription” as used herein, refers to the enzymatic synthesis of an RNA copy of one strand of DNA (i.e, template) catalyzed by an RNA polymerase (e.g. a DNA-dependent RNA polymerase). “Reverse transcription”, refers to the enzymatic synthesis of a DNA copy of one strand of RNA (i.e, template) catalyzed by an Reverse Transcriptase polymerase (e.g. a RNA-dependent DNA polymerase).

“Target analyte” or “target” as used herein, refers to any molecule, molecular complex, cell or other substance, without limitation, that it is desirable to detect in an assay, and which may be present in a sample. In certain aspects of the invention, nucleic acid targets can be tagged with biotin, which possess exceptional binding affinity to avidin and streptavidin, and can facilitate detection of target by methods of the present invention when the target-specific binding moiety coupled to a nanoparticle is avidin or streptavidin. The bond formed between biotin and avidin or streptavidin has the highest affinity of any known non-covalent bond with a K_{d} of approximately 10⁻¹⁴ to 10⁻¹⁵ M. Biotin is a small molecule that can be incorporated into DNA during PCR by using end labeled primers to give double stranded DNA products with biotins on one or both ends. Biotin can also be incorporated during PCR using biotinylated deoxyribonucleoside 5′-triphosphates (dNTPs), such as biotin-4-dUTP and an exonuclease-free thermostable DNA polymerase (e.g., Vent_{R}® (exo-), New England Biolabs, Ipswich, Mass.). See Tasara et al., Nucleic Acids Research, 2003, Vol. 31:2636-46, 2003. Replacement of dTTP with biotin-4-dUTP, in conjunction with an appropriate template, facilitates a high degree of target analyte biotinylation. Steptavidin and avidin are multimeric proteins that bind 4 molecules of biotin each. Furthermore, biotin is believed to bind cooperatively to streptavidin, thereby favoring binding of multiple biotin molecules to a single streptavidin and thus formation of higher order NPC-A complexes (discussed below). See Sano et al., J. Biol. Chem., 265: 3369-3373, 1990, which is incorporated herein by reference. In situations where a high ratio of biotin binding to streptavidin is not desirable, monovalent, divalent, trivalent and recombinant forms of streptavidin can be used. See Fairhead, et al. J. Mol. Biol. 426:199-214 (2014), which is incorporated herein by reference.

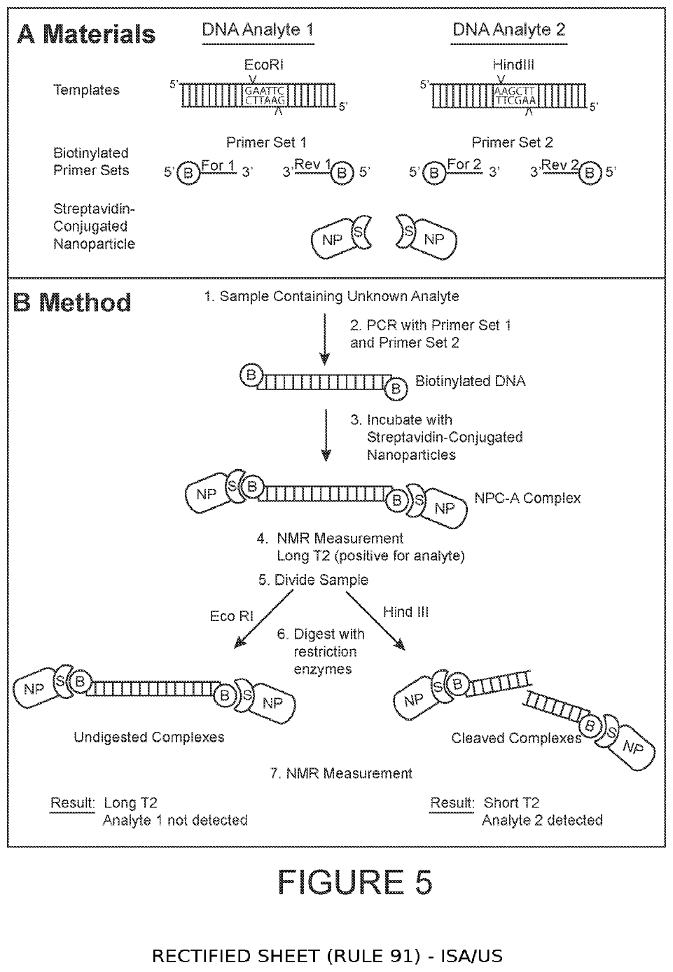

Thus, in one embodiment of the invention, which is illustrated in FIG. 5, the target analyte is a nucleic acid or the target is a cell or virus containing a nucleic acid, which is amplified using biotinylated primers.

Also contemplated by the term “target analyte” are species that are derived from a target analyte as surrogate for the intact target. For example, a virus can be detected by detecting a nucleic acid specific to the virus. Such surrogates (e.g., DNA and RNA), can be considered target analytes for the purposes of detecting the virus according to certain embodiments of the present invention. In such cases, detecting viral-specific nucleic acid is referred to as “detecting the virus”. The skilled artisan will appreciate the distinction between target analytes and surrogate molecules, including the inferences that can and cannot be made. For example, the detection of viral nucleic acid does not indicate whether a transmissible and replication-competent virus is present.

The term “sample” as used herein refers to an aliquot of material, frequently an aqueous solution or an aqueous suspension that may be derived from biological material and may be suspected of containing an target analyte. Samples to be assayed for the presence of an analyte by the methods of the present invention include, for example, cells, tissues, homogenates, lysates, extracts, purified or partially purified proteins and other biological molecules and mixtures thereof. Nonlimiting examples of samples typically used in the methods of the invention include human and animal body fluids such as whole blood, serum, plasma, cerebrospinal fluid, sputum, bronchial washings, bronchial aspirates, urine, lymph fluids and various external secretions of the respiratory, intestinal and genitourinary tracts, tears, saliva, milk, white blood cells, myelomas and the like; biological fluids such as cell culture supernatants; tissue specimens which may or may not be fixed; cell specimens which may or may not be fixed; environmental samples, such as air samples, soil samples, aerosols, hydrosols, wipe samples (wet or dry) obtained from surfaces (wet or dry), water or other fluids, complex samples such as food and clinical specimens such as blood, urine, saliva, tissue, sputum and stool.

The samples used in the methods of the present invention will vary based on the nature of the tissues, cells, extracts or other materials, especially biological materials, to be assayed. Methods for preparing e.g. homogenates, suspensions, solutions, extracts and fractions (including nucleic acid) from cells or other biological materials are well known in the art and can be readily adapted in order to obtain a sample that is compatible with the methods described herein.

“Amplification” as used herein, refers to the process of making identical copies of a polynucleotide, such as a DNA or RNA fragment or region. Amplification can be accomplished by LAMP polymerase chain reaction (PCR), but other methods known in the art may be suitable to amplify DNA fragments of the invention.

A “target DNA or RNA sequence” or “target DNA or RNA” is a DNA or RNA sequence of interest for which detection, characterization or quantification is desired. The actual nucleotide sequence of the target DNA may be known or not known. A “target DNA or RNA fragment” or “target fragment” is a segment of DNA or RNA containing the target DNA or RNA sequence. Target fragments can be isolated from a sample or produced by any method including e.g., shearing, sonication, or digestion with one or more restriction endonucleases.

As used herein, a “template” is a polynucleotide from which a complementary oligo- or polynucleotide copy is synthesized.

“Synthesis” generally refers to the process of producing a nucleic acid, via chemical or enzymatic means. Chemical synthesis is typically used for producing single strands of a nucleic acid that can be used and primers and probes. Enzyme-mediated “synthesis” encompasses both transcription and replication from a template. Synthesis includes making a single copy or multiple copies of the target. “Multiple copies” means at least 2 copies. A “copy” does not necessarily mean perfect sequence complementarity or identity with the template sequence. For example, copies can include nucleotide analogs, intentional sequence alterations (such as sequence alterations introduced through a primer comprising a sequence that is hybridizable, but not complementary, to the template), and/or sequence errors that occur during synthesis.

The terms “polynucleotide” and “nucleic acid (molecule)” are used interchangeably to refer to polymeric forms of nucleotides of any length. The polynucleotides may contain deoxyribonucleotides, ribonucleotides and/or their analogs. Nucleotides may be modified or unmodified and have any three-dimensional structure, and may perform any function, known or unknown. The term “polynucleotide” includes single-stranded, double-stranded and triple helical molecules. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers.

“Oligonucleotide” refers to polynucleotides of between 2 and about 100 nucleotides of single- or double-stranded nucleic acid, typically DNA. Oligonucleotides are also known as oligomers or oligos and may be isolated from genes and other biological materials or chemically synthesized by methods known in the art. A “primer” refers to an oligonucleotide containing at least 6 nucleotides, usually single-stranded, that provides a 3′-hydroxyl end for the initiation of enzyme-mediated nucleic acid synthesis. A “polynucleotide probe” or “probe” is a polynucleotide that specifically hybridizes to a complementary polynucleotide sequence. As used herein, “specifically binds” or “specifically hybridizes” refers to the binding, duplexing, or hybridizing of a molecule to another molecule under the given conditions. Thus, a probe or primer “specifically hybridizes” only to its intended target polynucleotide under the given binding conditions, and an antibody “specifically binds” only to its intended target antigen under the given binding conditions. The given conditions are those indicated for binding or hybridization, and include buffer, ionic strength, temperature and other factors that are well within the knowledge of the skilled artisan. The skilled artisan will also be knowledgeable about conditions under which specific binding can be disrupted or dissociated, thus eluting or melting e.g, antibody-antigen, receptor-ligand and primer-target polynucleotide combinations.

“Nucleic acid sequence” refers to the sequence of nucleotide bases in an oligonucleotide or polynucleotide, such as DNA or RNA. For double-strand molecules, a single-strand may be used to represent both strands, the complementary stand being inferred by Watson-Crick base pairing.

The terms “complementary” or “complementarity” are used in reference to a first polynucleotide (which may be an oligonucleotide) which is in “antiparallel association” with a second polynucleotide (which also may be an oligonucleotide). As used herein, the term “antiparallel association” refers to the alignment of two polynucleotides such that individual nucleotides or bases of the two associated polynucleotides are paired substantially in accordance with Watson-Crick base-pairing rules. Complementarity may be “partial”, in which only some of the polynucleotides' bases are matched according to the base pairing rules. Or, there may be “complete” or “total” complementarity between the polynucleotides. Those skilled in the art of nucleic acid technology can determine duplex stability empirically by considering a number of variables, including, for example, the length of the first polynucleotide, which may be an oligonucleotide, the base composition and sequence of the first polynucleotide, and the ionic strength and incidence of mismatched base pairs.

As used herein, the term “hybridization” is used in reference to the base-pairing of complementary nucleic acids, including polynucleotides and oligonucleotides containing 6 or more nucleotides. Hybridization and the strength of hybridization (i.e., the strength of the association between the nucleic acids) is impacted by such factors as the degree of complementary between the nucleic acids, the stringency of the reaction conditions involved, the melting temperature (Tm) of the formed hybrid, and the G:C ratio within the duplex nucleic acid. Generally, “hybridization” methods involve annealing a complementary polynucleotide to a target nucleic acid (i.e., the sequence to be detected either by direct or indirect means). The ability of two polynucleotides and/or oligonucleotides containing complementary sequences to locate each other and anneal to one another through base pairing interactions is a well-recognized phenomenon.

When non-specific hybridization of nucleic acid is observed, reactions can be carried out at higher stringency, such as medium or high stringency instead of low stringency. As used herein “high stringency” refers to hybridization in 0.2-0.5× SSC at 60-65° C., or the conditions that requires 100% complementarity between two single strand nucleic acids for duplex formation to occur; “medium stringency” refers to 1-2× SSC at 50-60° C., and “low stringency” refers to 4-6× SSC at 40-50° C., where 1× SSC is 0.15 M NaCl, 0.015 M Na citrate. Some degree of mismatch between the nucleic acids is tolerated at medium and low stringency. The skilled artisan will also be familiar with other conditions and agents that can be used to increase or decrease stringency of nucleic acid hybridization, such as increased temperature, addition of formamide or selecting nucleic acid analytes with higher GC content.

A “complex” is an assembly of components. A complex may or may not be stable and may be directly or indirectly detected. For example, as described herein, given certain components of a reaction and the type of product(s) of the reaction, the existence of a complex can be inferred. For example, in the abortive transcription method described herein, a complex is generally an intermediate with respect to a final reiterative synthesis product, such as a final abortive transcription or replication product.

As used herein, the term “contacting” (i.e., contacting a target analyte with nanoparticles) refers generally to providing access of one component, reagent, target analyte or sample to another. For example, contacting can involve mixing a solution comprising magnetic nanoparticles with a sample comprising a target analyte. “Contacting” is intended to include incubating target and nanoparticles together in vitro (e.g., adding nanoparticles to a liquid sample containing the target analyte).

As used herein, the term “substantially” refers to a great extent or degree. For example, “substantially uniform” can be used to describe a suspension in which nanoparticles are dispersed throughout a liquid to great extent or degree, although slight variations in the distribution of the nanoparticles may be present in different locations within the suspension. In other contexts, the term “substantially” when combined with other terms, means at least 80%, at least 85%, at least 90%, at least 95%, or at least 99%. For example, by “substantially free” (e.g., “substantially free from contaminants,” “substantially free of target analyte,” “substantially free of nanoparticles” or equivalent expressions), it is meant that the solution, liquid, sample or the like, is at least 80%, at least 85%, at least 90%, at least 95%, at least 99% or at least about 100% free of the referenced material, e.g., contaminants, target analyte, nanoparticles or equivalent thereof. In contrast, a “substantially similar” composition, process, method, solution, or the like, is at least 80%, at least 85%, at least 90%, at least 95%, or at least 99% similar to the reference composition, process, method, solution or the like previously described herein, or in a previously described process or method incorporated herein by reference in its entirety.

“Binding moieties,” as used herein, refers to specific groups of atoms within a molecule that are responsible for the characteristic binding of a molecule to another molecule. Binding moieties are a type of functional group that can be conjugated to or integrated in a variety of base molecules while retaining their functional property, e.g., binding to another molecule. In particular, “target analyte-specific binding moieties” bind to their cognate target analytes and impart target-specific binding to a base molecule, such as a nanoparticle, when integrated in or conjugated thereto. Target-specific binding moieties suitable for use in the methods of the present invention include, but are not limited to, naturally occurring and synthetic antibodies, nucleic acids, peptides, polypeptides, proteins, small organic molecules, ligands, receptors, carbohydrates (e.g., monosaccharides, polysaccharides, and sugars), aptamers and combinations thereof.

The terms “detect”, “detection”, “detecting” and “detected” as used herein have their ordinary meaning as known to those skilled in the art and refer to identifying the presence, absence or amount of the molecule (e.g., a nucleic acid) to be detected.

“Microarray” and “array,” are used interchangeably to refer to an arrangement of a collection of compounds, samples, or molecules such as oligo- or polynucleotides. Arrays are typically “addressable” such that individual members of the collection have a unique, identifiable position within the arrangement. Arrays can be formed on a solid substrate, such as a glass slide, or on a semi-solid substrate, such as nitrocellulose membrane, or in vessels, such as tubes or microtiter plate wells. Typical arrangements for an array are 8-row by 12-column configuration, such as with a 96-well microtiter plate, and 16-row by 24-column configuration, such as with a 384-well PCR plate However, other arrangements suitable for use in the methods of the present invention will be well within the knowledge of the skilled artisan.

The term “solid support” refers to any solid phase that can be used to immobilize e.g., a capture probe or other oligo- or polynucleotide, a polypeptide, an antibody or other desired molecule or complex. Suitable solid supports will be well known in the art and include, but are not limited to, the walls of wells of a reaction tray, such as a microtiter plate, the walls of test tubes, polystyrene beads, paramagnetic or non-magnetic beads, glass slides, nitrocellulose membranes, nylon membranes, and microparticles such as latex particles. Typical materials for solid supports include, but are not limited to, polyvinyl chloride (PVC), polystytrene, cellulose, agarose, dextran, glass, nylon, latex and derivatives thereof. Further, the solid support may be coated, derivatized or otherwise modified to promote adhesion of the desired molecules and/or to deter non-specific binding or other undesired interactions. The choice of a specific “solid phase” is usually not critical and can be selected by one skilled in the art depending on the methods and assays employed. Conveniently, the solid support can be selected to accommodate various detection methods. For example, 96 or 384 well plates can be used for assays that will be automated, for example by robotic workstations, and/or those that will be detected using, for example, a plate reader.

Suitable methods for immobilizing molecules on solid phases include ionic, hydrophobic, covalent interactions and the like, and combinations thereof. However, the method of immobilization is not typically important, and may involve uncharacterized adsorption mechanisms. A “solid support” as used herein, may thus refer to any material which is insoluble, or can be made insoluble by a subsequent reaction. The solid support can be chosen for its intrinsic ability to attract and immobilize a capture reagent. Alternatively, the solid support can retain additional molecules which have the ability to attract and immobilize e.g., a “capture” reagent. “Limit of detection”, “threshold” and “detection threshold” are used interchangeably to refer to the lowest quantity or concentration of an target that can be accurately detected.

As used herein, “epidemic”, refers to a communicable disease (i.e., transmissible, infectious and/or contagious) that affects a large number of people within a community, population, or region; “pandemic” refers to an epidemic that has spread over multiple countries or continents; and “endemic” refers to a communicable disease that is limited to a particular population or geographic region such as a state or country.

The invention is based on the observations that magnetic fields can be used to detect nanoparticles and can also be exploited to influence the movement and interactions of nanoparticles, including nanoparticles coupled to target-specific binding moieties to form “target analyte-specific nanoparticles”. In particular, magnetic fields can be used to move nanoparticles, concentrate nanoparticles and facilitate aggregation of nanoparticles. This in turn can be used to influence the binding of the target-specific moieties on target-specific nanoparticles with their cognate target and thus form target-specific nanoparticle complexes in the presence of the target.

The present invention provides methods to rapidly detect target analytes with very high sensitivity, thereby obtaining a detectable signal within hours, and in many cases within 30 to 60 minutes, even when the target analyte highly dilute. The invention is based on the observations that magnetic fields can be used to detect nanoparticles and can also be exploited to influence the movement and interactions of nanoparticles, including nanoparticles coupled to target analyte-specific binding moieties to form “target analyte-specific nanoparticles”. In particular, magnetic fields can be used to move nanoparticles, concentrate nanoparticles and facilitate aggregation of nanoparticles. This in turn can be used to influence the binding of the target-specific moieties on target analyte-specific nanoparticles with their cognate targets and thus form target-specific nanoparticle complexes in the presence of target.

As is known in the art, and discussed in detail below, aggregation of nanoparticles can be detected using NMR technology. According to the present invention, the NMR signal of a sample containing target-specific nanoparticle complexes with target can be increased and optimized by subjecting the sample to a controlled magnetic concentration, disruption and resuspension process.

Thus, the present invention provides methods for target analyte detection based on magnetic resonance measurements. The methods use “nanoparticles”, which are nanometer-scale particles having a paramagnetic or superparamagnetic component, typically encased in a nonmagnetic shell, which are conjugated to moieties that bind directly or indirectly to an analyte (“target analyte-specific binding moieties”). In one aspect of the invention, target analytes are detected using magnetic nanoparticles in the form of magnetic resonance “nanoswitches”, i.e., magnetic nanoparticles that are induced to switch between dispersed and clustered states by target analytes or various forms of target analytes, with accompanying changes in the spin-spin relaxation time (i.e, spin-spin dephasing time or T2) of adjacent water molecules. In such embodiments, the T2 value, which can be measured, is dependent on the degree of aggregation of the nanoparticles in the sample.

### Nuclear Magnetic Resonance

“Nuclear magnetic resonance” or “NMR” is a physical phenomenon in which atomic nuclei in a magnetic field absorb and re-emit or “resonate” electromagnetic radiation. Atomic nuclei that contain an odd number of protons and/or of neutrons have an intrinsic magnetic moment and angular momentum, which is referred to as “non-zero spin”, and are NMR-active. Atomic nuclei with an even number of protons and neutrons have a total spin of zero and are thus NMR-inactive. Advantageously, the hydrogen in a water molecule has a nucleus with non-zero spin and thus NMR analysis of water-soluble molecules is frequently performed in an aqueous solution.

When placed in a magnetic field, NMR-active nuclei (i.e., “non-zero spin nuclei”) absorb electromagnetic radiation at a frequency characteristic of the isotope. Resonant absorption by nuclear spins will occur only when the electromagnetic radiation of the correct frequency (i.e., the Larmor frequency for the isotope) is used. The phenomenon of nuclear magnetic resonance makes the magnetization measurable. The resonance frequency and intensity are directly proportional to the strength of the magnetic field.

NMR based technologies typically include a series of sequential steps:
1. Alignment (“polarization”) of the magnetic nuclear spins in an applied, spatially and temporally constant (“uniform”) magnetic field;
2. Perturbation of this alignment (“excitation”) of the nuclear spins by applying an electromagnetic, typically radio frequency (RF) pulse; and
3. Measurement of a resonance energy “signal”.

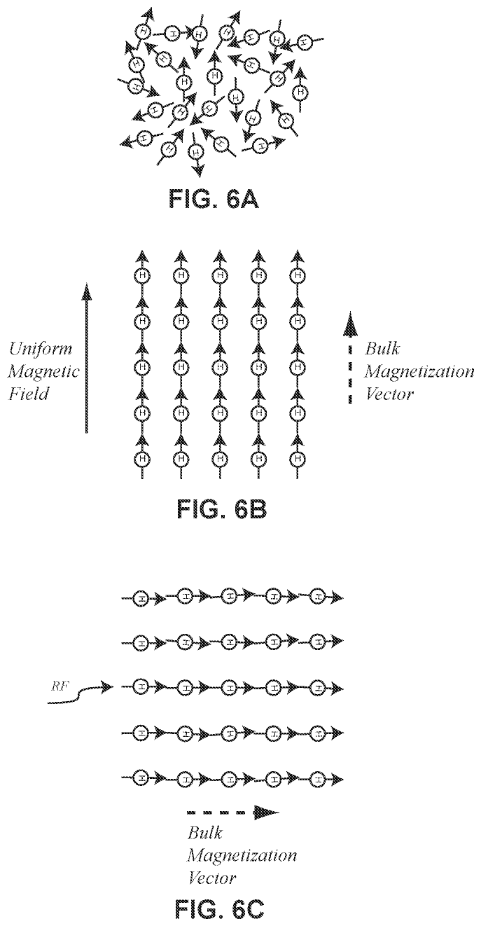

In an unmagnetized sample containing non-zero spin atoms, the spins of NMR-active nuclei are randomly oriented. See FIG. 6A. Polarization in the presence of a uniform magnetic field causes the NMR-active nuclei to align with the direction of the uniform magnetic field. See FIG. 6E. As used herein, “uniform magnetic field” refers to one which has the same magnitude and direction throughout a volume, such as the sample volume. It should be noted that a uniform magnetic field alters the spin orientation of the nuclei, but does not move magnetizable atoms as a non-uniform magnetic field will. Although the polarization of an individual magnetic nuclear spin cannot be easily detected, the magnetic moment of the alignment is a vector quantity that is additive for detectable non-zero spin nuclei in a sample. Thus, the sum of these vector quantities in the sample (the “bulk magnetization vector” or “bulk magnetization”; broken arrow, FIG. 6B) is measured using a detector.

Upon application of suitable electromagnetic energy, the spins of individual atomic nuclei vectors are perturbed, and thus the bulk magnetization vector reorients. A suitable electromagnetic pulse is typically a radio frequency (RF) pulse at the Lamor frequency for the relevant non-zero spin nuclei (e.g., H), and is typically applied in a plane perpendicular to the direction of uniform magnetic field. The Lamor frequency of H is 24.58 MHz/T. When the duration of the pulse is sufficient, it causes the bulk magnetization vector to reorient into a plane perpendicular to the uniform magnetic field as illustrated in FIG. 6C. The bulk magnetization vector can be detected and recorded over time as it reorients in response to the RF pulse, or at a single time point once reorientation has occurred.

### Relaxation

Following a perturbation of the alignment (excitation of non-zero spin nuclei, the bulk magnetization vector recovers to its original steady state over time; this process is referred to as nuclear “magnetic relaxation.” Two fundamental time constants are used to describe this relaxation in terms of a single-exponential process: T₁ and T₂.

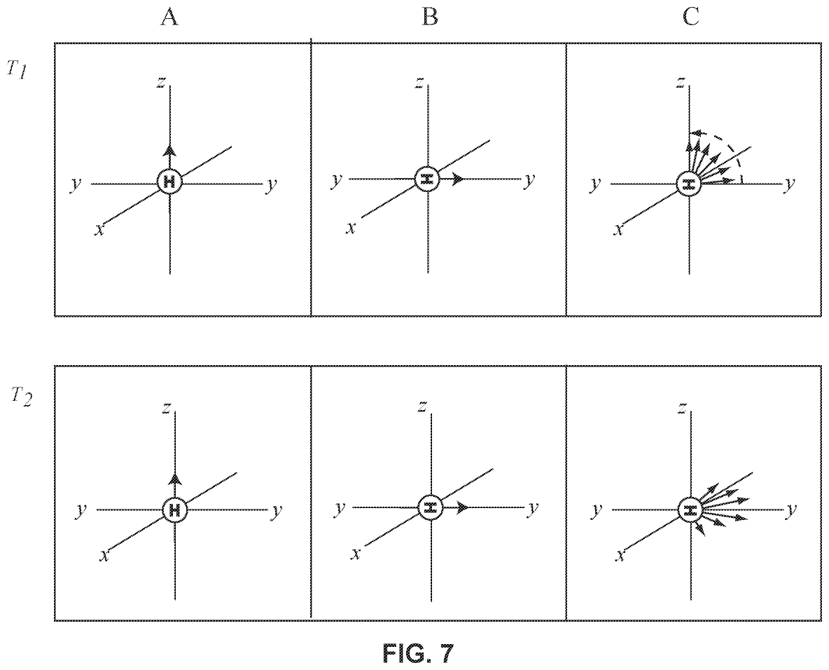

T₁—Longitudinal or Spin-Lattice Relaxation. Recovery of the bulk magnetization vector along the direction of the first magnetic field (z axis) (see FIG. 7, top panel) is described by the “spin-lattice relaxation time” or “longitudinal relaxation time” (T₁). T₁ quantifies the rate of transfer of energy from the nuclear spin system to its surroundings, including neighboring atoms atom and molecules (the “lattice”). T₁ measurements allow structural characterization of complex biological molecules using NMR, where transfer of energy to the lattice is affected by the nature of surrounding atoms in the molecule. Typically, T₁ is on the order of milliseconds to seconds. T₁ can be detected by using a detector coil to observe changes in the bulk magnetization vector of a sample as a function of the time between a depolarization pulse and a polarization measurement.
T₂—Transverse or Spin-Spin Relaxation. Recovery of the bulk magnetization vector in the plane perpendicular to the direction of the first magnetic field (xy plane) (see FIG. 7, bottom panel) is described by the “spin-spin relaxation time” or “transverse relaxation time” (T₂). T₂ quantifies the rate of transfer of energy between spins lying partially in the transverse plane without energy transfer to the lattice. For aqueous signals, T₂ is generally in the range of 100 milliseconds or more. Solid samples on the other hand, generally have T₂ values in the range of 1 to 100 microseconds.

Spin-spin relaxation is typically measured by a series of RF pulses to give rise to “spin echo signals”. A spin echo is generated by a 90-degree pulse followed by a small delay time (τ), followed by a 180° pulse)(90°-τ-180°. A second i, identical in time to the first, is used before the bulk magnetization vector is recorded. This series of RF pulses and time delays is used to first dephase the nuclear magnetic moments comprising the bulk magnetization in the plane perpendicular to the first magnetic field during the first τ, and refocus the remaining bulk magnetization in this plane during the second τ. This latter refocusing creates an echo signal, which can be detected and recorded. The most common method to measure spin-spin relaxation is that originally described by Carr and Purcell (“Effects of Diffusion on Free Precession in Nuclear Magnetic Resonance Experiments”, Physical Rev. 94:630-638 (1954)), which is a modification of the method described by Meiboom and Gill (“Modified Spin-Echo Method for Measuring Nuclear Relaxation Times”, Rev. Scientific Instruments 29: 688-691 (1958)), which is incorporated herein by reference. The Can-Purcell modified Meiboom-Gill (CPMG) method uses a series of small time delays followed by 180-degree pulses after the initial 90°-τ-180° sequence described above. This in turn is followed by the resultant bulk magnetization vector—[90×−(τ−180y−τ−record)n]. The amplitudes of the spin echo signals are proportional to the bulk magnetization remaining at the time of the echo, which becomes successively smaller as the time increases (typically, as the value of n increases). Therefore, measuring the amplitude of the bulk magnetization vector after various values of n and fitting the data to a single exponential decay with T₂ as the relaxation time provides a direct measure of T₂.

Instruments and software suitable for performing NMR and determining relaxation constants (e.g. T₁ and T₂) are available from various vendors including Bruker Corp. (Tucson, Ariz.; e.g. Minispec, NanoBay and AVANCE NMR with Dynamics Center software), HTS-110 Limited (Lower Hutt, N Z; e.g. Field Cycling Relaxometer), Magritek (San Diego, Calif.; e.g. Kea NMR), Oxford Instruments (Austin, Tex.; e.g., Pulsar with SpinFlow software). Typically, an NMR spectrometer operating at 20 MHz can be used. The sample coil should be small enough to fill a 100 microliter sample with filling factor between 50 to 100% A CPMG (Car-Purcell-Meiboom-Gill) pulse sequence can be used to measure T₂ with an RF field of 20 MHz.

### MRI Versus NMR

MRI exploits the same physical effects as NMR spectroscopy, which is typically used to identity unknown compounds by the resonant properties of the atoms that comprise it. In both NMR and MRI a sample is placed in a static magnetic field, briefly excited with radio-frequency energy, and then allowed to re-emit that energy. NMR detects the characteristic frequency of the re-emitted energy, which varies very slightly based on the structure of the molecule. MRI focuses on the disruption of protons (prevalent in water in the human body) aligned in a magnetic, and their re-alignment following the radiofrequency excitation, which change based on the thickness and hardness of the tissue the protons/water molecules are in. Carefully monitoring of the arrival of re-emitted photons in the MRI's detectors allows the locations and shapes of different tissues to be identified. MRI devices have been developed to generate and analyze such shapes and locations in 3-D. and thus are well-suited to analyzing samples in 3-D arrays.

The main difference between NMR spectroscopy and MRI imaging is that NMR generates information (a spectrum of light corresponding to chemical structure) based on the frequency of emitted radiation (which is related to the speed of the jiggling protons). MRI instead generates information (images of the body) using the intensity of radiation (the quantity of re-emitted photons) arriving from various parts of body. Protons in dense or solid structures tend to be more or less prone to misalignment when the disrupting radio waves are applied to the body's tissue, resulting in a lower number of re-emitted photons coming from that region and thus a darker area in the resulting image.

### Magnetic Resonance Effects of Nanoparticles

In an aqueous medium, which generally has a long T₂ (on the order of 2,000 msec), the addition of nanoparticles generally reduces the magnetic resonance property T₂ when the nanoparticles are uniformly dispersed or suspended throughout the solution. This effect is thought to result from depolarization of adjacent water molecules, with each nanoparticle acting as a “depolarizing center”. However, if the nanoparticles (of the types and sizes specified herein) in an aqueous environment become clustered through agglomeration, aggregation, concentration, precipitation or the like, the number of depolarizing centers is reduced, and therefore the effect on T₂ is also reduced, with the result that the T₂ of the aqueous solution is increased relative to the T₂ of the medium when the nanoparticles are dispersed. Typically, in methods of the invention, the increase in T₂ is proportional to the reduction in depolarizing centers, which is in turn dependent on aggregation of nanoparticles.

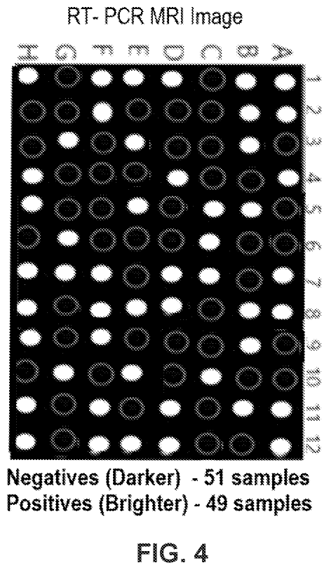

This effect can be exploited for the detection of target(s) in solution when the aggregation of nanoparticles is dependent on the interaction of nanoparticles, particularly target-specific nanoparticles, with target: in the absence of target, nanoparticles are dispersed, resulting in an overall depolarization of the water in the solution, and therefore, a short T₂ is observed; in the presence of target, target-specific nanoparticles form complexes with target, reducing the number of depolarizing centers, and thus overall depolarization is reduced (relative to the suspension of dispersed nanoparticles) and T₂ increases. This effect is illustrated in FIG. 4.

Furthermore, nanoparticles of the invention are attracted by magnetic fields and can thus be concentrated to the region of highest magnetic field strength in a non-uniform magnetic field. As used herein, the term “non-uniform magnetic field” refers to one in which the magnetic field changes from one place to another. Typically, a non-uniform magnetic field is strongest closest to the source of the magnetic field (i.e. magnet), and decreases in magnitude as the distance from the source increases (c.f., “uniform magnetic field” in which the magnetic field has the same magnitude and direction throughout a region).

Paramagnetic materials, such as the nanoparticles used in the methods of the invention, move in a non-uniform magnetic field, but not in a uniform magnetic field. When an external magnet is contacted with a test tube containing a suspension of nanoparticles in a liquid, for example, and thereby creates a non-uniform magnetic field, the nanoparticles will be drawn toward the external magnet and concentrate of the inside surface of the tube adjacent to the magnet, as shown in FIG. 4 (tube D). Since the effect is dependent on the presence of the external magnet, removing the magnet releases the nanoparticles. The concentrated nanoparticles will eventually redistribute by diffusion to become dispersed throughout the liquid, and the process of redistribution can be accelerated by resuspension, such as by mixing, stirring, scraping and the like, as described below in greater detail.

### Properties of Nanoparticles

Nanoparticles according to the present invention can be monodisperse (a single crystal of a magnetic material, e.g., metal oxide, such as superparamagnetic iron oxide, per nanoparticle) or polydisperse (a plurality of crystals, e.g., 2, 3, or 4 crystals per nanoparticle). The magnetic metal oxide can also include cobalt, magnesium, zinc, silicon, tin, titanium, bismuth, and/or or mixtures of these metals with iron. Important features and properties of nanoparticles that are contemplated for use in the methods of the invention include: (i) high relaxivity, i.e., strong effect on water relaxation; (ii) sufficiently small size that the nanoparticles remain suspended in an aqueous solution; (iii) sufficiently large size that the nanoparticles can be concentrated using a magnet under conditions used in the assay methods of the invention; and (iv) stability in solution, i.e., the nanoparticles neither dissolve nor precipitate.

When used in certain embodiments of the invention, nanoparticles of 100-200 nm have been found to be very effective at depolarizing neighboring water molecules, resulting in a reduction of T₂ from about 2,000 msec. to about 100 to about 200 msec. Moreover, this size can be readily concentrated by a non-uniform magnetic field and remain in suspension for extended periods of time without precipitating. Advantageously, non-specific binding to smaller nanoparticles may also be minimized. Nanoparticles in the range of at least about 60 nm to about 500 mn, such as about 75 nm, about 100 nm, about 125 nm, about 150 nm, about 175 nm, about 200 nm, about 225 nm, about 250 nm, about 275 nm, about 250 nm, about 300 nm, about 325 nm, about 350 nm, about 375 nm, 400 nm, about 425 nm, about 450 nm, about 450 and 500 nm or more are contemplated for use in the methods of the invention. Particularly suitable nanoparticles sizes include at least about 80 nm to about 180 nm, such as about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, and about 175 nm.

### Preparation of Nanoparticles

Synthesis of a variety of polymer coated metal oxide “core” particles for biological applications have been disclosed in the prior art, particularly for magnetic resonance imaging applications. Examples include magnetizable nanoparticles below 200 that are suitable for use in the methods of the present invention.

Nanoparticle cores may be prepared using known techniques from any type of magnetizable (e.g. paramagnetic, or superparamagnetic) material, including Fe, Co, Sn, Al, Ni, Ti, Bi, Zr, Pt, Pd, Sr, Ir, Ca, Mg, Mn, Ba and/or Zn. In certain embodiments, nanoparticles are prepared from metal oxides, such as iron oxides, e.g. magnetite (Fe₃O₄) and maghaemite (γFe₂O₃), pure metals, such as Fe and Co, as well as alloys, such as and FePt, using a variety of methods including co-precipitation, thermal decomposition and/or reduction, micelle synthesis, hydrothermal synthesis, plasma based and laser pyrolysis techniques. See Lu et al., Angewandte Chemie Int'l Ed. 46:1222-44, 2007), which is incorporated herein by reference. Co-precipitation can be used to synthesize iron oxides such as Fe₃O₄ and γFe₂O₃ from aqueous Fe²⁺/Fe³⁺ salt solutions. For example, PCT Int'l Pub. No. WO 2006/125452, which is incorporated herein by reference) discloses the preparation of iron oxide particles by alkaline coprecipitation of ferric and ferrous chlorides in aqueous solution.

Monodisperse magnetic nanocrystals can be synthesized through the thermal decomposition of organometallic compounds in high-boiling organic solvents containing stabilizing surfactants. See U.S. Pat. No. 7,029,514, which is incorporated herein by reference. In another approach, water-in-oil microemulsions containing the desired reactants, can be mixed and will coalesce to form micelles with precipitates therein. Such microemulsions can thus be used as nanoreactors to form of nanoparticles. See e.g., U.S. Pat. No. 8,512,665, which is incorporated herein by reference. A variety of different nanocrystals can be synthesized by hydrothermal synthesis, which exploits the general phase transfer and separation mechanism occurring at the interfaces of the liquid, solid, and solution phases present during the synthesis. See e.g., U.S. Pat. No. 7,732,015 which is incorporated herein by reference. U.S. Pat. Pub. No. 2004/0009118, which is incorporated herein by reference discloses methods for producing metal oxide nanoparticles by generating an aerosol of solid metallic microparticles, generating plasma with a plasma hot zone at a temperature sufficiently high to vaporize the microparticles into metal vapor, and directing the aerosol into the hot zone of the plasma.

Additional magnetic particles that may be suitable for use in the methods of the present invention are disclosed in, U.S. Pat. Nos. 4,554,088, 5,055,288, 5,262,176, 5,512,439, and 7,459,145, and U.S. Pat. Pub. Nos. 2003/0092029, 2003/0124194, 2006/0269965, and 2008/0305048, the disclosures of which are incorporated herein by reference in their entirety.

Suspensions of nanoparticles produced using the techniques described hereinabove typically contain particles with varying characteristics such as size. The non-homogeneous nature of nanoparticle suspensions may degrade the performance of the suspension in methods of the present invention. Methods to improve the homogeneity of the particles have been described for the production of dispersed aqueous systems that are intended for use as injectable fluids. Such homogenization methods include the rotor-stator- and high-pressure-methods. The use of liquid-jet- or liquid-slot-nozzle-high-pressure homogenization machines (e.g., available from Microfluidics, a division of MFIC, Corp., Newton, Mass.) enables high mechanical energy deposition (e.g., U.S. Pat. Nos. 5,635,206; 5,595,687, which is incorporated herein by reference).

High-pressure homogenization machines for the production of metal oxide nanoparticle compositions using controlled coalescence followed by drying in emulsion, in which the non-aqueous component contains an oxide have been reported in conjunction with industrial production of catalytic materials (e.g., U.S. Pat. No. 5,304,364, which is incorporated herein by reference) as well as electrographic pigment particles, ceramic powders, felt materials, spray layers, active substances carriers, and ion exchange resins (e.g., U.S. Pat. No. 5,580,692, which are incorporated herein by reference). An emulsion is the dispersion of multi-phase systems of two or more insoluble liquids. Emulsions consist of at least one continuous (outer) phase (e.g. water) and one isolated (dispersed or inner) phase (e.g., oil). Emulsions are thermodynamically unstable. High-pressure homogenization (HPH) is often used for the preparation or stabilization of emulsions and suspensions in pharmaceutical, cosmetic, chemical and food industries. It is also known that nano-scale metal oxides can be prepared using high shear forces in a fluidizing apparatus (e.g., U.S. Pat. No. 5,417,956, which are incorporated herein by reference). For some applications, pressures up to 200 Mega Pa (MPa) or higher are used.

Typically, the nanoparticle core is coated with a polymer or other substance to stabilize the core and to provide a material to which target-specific binding moieties can be conjugated. The term “conjugate” or “conjugated” as used herein refers to two molecules that have been covalently attached or otherwise linked together. Conjugation to appropriate surface coatings can be performed by methods that are well known in the art, such as those described in Wong, et al., Chemistry of Protein and Nucleic Acid Cross-Linking and Conjugation, 2^{nd} Ed. (CRC Press; Boca Raton, Fla.) and Hermanson, “Bioconjugate Techniques, 3^{rd} Ed.” Academic Press, New York, 2013, which are incorporated herein by reference. Generally, the ratio of binding moiety to nanoparticles can be controlled to give a desired proportion of nanoparticles to binding moieties.

The most commonly used coating material is dextran in its various forms. Also used are other compatible carbohydrates, such as arabinogalactan, starch, glycosaminoglycan, proteins (e.g., U.S. Pat. No. 6,576,221, which is incorporated herein by reference), and silica. One such method is the precipitation of Fe(II)- and Fe(III)-salts in the presence of dextran (e.g., U.S. Pat. No. 4,452,773, which is incorporated herein by reference). A modified method involves the use of ultrasound treatment followed by thermal treatment in the same apparatus (e.g., U.S. Pat. No. 4,827,945, which is incorporated herein by reference). The quality of this method is enhanced by magnetic classification (e.g., PCT Int'l Pub. No. WO 90/07380, which is incorporated herein by reference). Further encapsulation/coating may improve the properties of the nanoparticles due to the use of amphiphilic substances for stabilization. (see e.g., U.S. Pat. No. 5,545,395; and European Patent No. EP 0272091, which are incorporated herein by reference).

Wet chemical synthesis can be preceded by a coating with polymer components (Core-shell-Method), or it can be performed in the presence of the polymer (One-pot-Method). For the core-shell-method, it is necessary to add stabilizing substances to the iron oxide, because iron oxide cores tend to aggregate in aqueous solutions. Amphiphilic substances (e.g., PCT Int'l Pub. No. WO 01/56546, which is incorporated herein by reference) or additional nanoparticles with electrically charged surfaces can be selected as stabilizers (e.g., U.S. Pat. No. 4,280,918, which is incorporated herein by reference). However, surface-active substances used as stabilizers may influence and limit the functionality of the nanoparticle surface. The One-pot method involves the coating of the polymer directly during the formation of the iron oxide to stabilize the formation process and the growth of the crystals from the solution.

In certain embodiments, the coating will be functionalized to facilitate conjugation to target-specific binding moieties. Functional groups contemplated for use in the nanoparticle coatings of the present invention include, but are not limited to: amino, carboxyl, sulfhydryl, amine, imine, epoxy, hydroxyl, thiol, acrylate, and/or isocyano groups. Carboxy functionalized nanoparticles can be made, for example, according to the method of Gorman (see PCT Int'l Pub. No. WO 00/61191). More stably coated and amino-functionalized nanosensors can be prepared, for example, by cross-linking a dextran coating of a metal oxide particle core with epichlorohydrin, then treating with ammonia to provide functional amino groups. Aminated cross-linked iron oxide nanoparticles (amino-CLIO) have been made with 40 amino groups per particle, with an average particle size from about 40 to about 50 nm. These particles can withstand harsh treatment, such as incubation at 120° C. for 30 minutes, without a change in size or loss of their dextran coat. Amino groups can react by N-hydroxysuccinimide (NHS) based

Nanoparticles of various sizes and compositions that are suitable for use in the methods of the present may be also be obtained commercially, for example from: Sigma Aldrich (St. Louis, Mo.), Nanocs Inc. (NY, N.Y.), NanoComposix (San Diego, Calif.), MKNano (Missisauga, ON, Canada), Miltenyi Biotec (San Diego, Calif.), Ademtech (Pessac, France), Chemicell GmbH (Berlin, Germany), Corpuscular Inc. (Cold Spring, N.Y.), Nvigen Inc. (Sunnyvale, Calif.), NanoTherics Ltd. (Staffordshire UK), Cytodiagnostics Inc. (Burlington, Ontario, Canada), Eprui Nanoparticles & Microspheres Co. (Nanjing, China), Polysciences, Inc®, SA. (Warrington, Pa.), and the like. Commercially available nanoparticles included those that are coated, uncoated, and functionalized as well as nanoparticles conjugated to common binding moieties such as biotin, avidin, streptavidin, second antibodies, Protein A, Protein G and the like.

### Target Nucleic Acid Detection Assay

In certain aspects, the invention is based on the observation that the reaction between target-specific nanoparticles and their targets can be influenced by and detected using magnetic fields. Methods are provided for detecting an target whereby target-specific nanoparticles specifically bind to the target, thereby forming nanoparticle-target complexes. A magnetic field is applied to the complexes in an aqueous solution, thereby magnetizing the nanoparticles. The applied magnetic field, if non-uniform, exerts forces on the nanoparticles. Nanoparticle-target complexes move in response to the applied magnetic force and the resultant magnetic force of adjacent nanoparticles. Furthermore, the interactions between nanoparticles and complexes thereof can be enhanced by the movement of the nanoparticles in response to the applied magnetic field.

Magnetic resonance signals are elicited from a sample comprising the complexes and the known liquid, by exposing the sample to radiofrequency (RF) radiation. A magnetic resonance property of the sample, such as T₂, can be determined from the magnetic resonance signals. Presence of the target can be detected by comparing the magnetic resonance property of the sample with the magnetic resonance properties of measured or known control samples and standards.

Advantageously, methods of the invention permit detection of target nucleic acids with very high specificity, despite near-neighbor interference, dirt, clutter, biological interferents such as cellular debris, proteinaceous interferents, paramagnetic interferents such as hemoglobin and humic acid (containing chelated iron), and the like.

The methods of the invention are useful for detection of target nucleic acids present in a liquid medium or in other forms, including but not limited to, aerosols, hydrosols, complex media such as food, and clinical specimens such as blood, urine, saliva, tissue, sputum and stool.

The liquid in which target nucleic acids are analyzed can be any fluid material that includes a nucleus having non-zero spin. Only nuclei with non-zero spin give rise to the NMR phenomena. The liquid includes such nuclei when molecules in the liquid, such as the hydrogen in a water molecule, have a nucleus with non-zero spin. Alternatively, the liquid may include non-zero spin nuclei as solutes or suspensions, such as a fluoridated solute which generates magnetic resonance signals at the ¹⁹F Larmor frequency. However, for the purposes of this invention, the liquid is typically water or an aqueous solution, and unless otherwise indicated, the terms “liquid”, “fluid”, “known liquid”, “liquid medium” and “solution” refer to water or an aqueous solutions.

The present invention provides methods provides for pathogen detection that combines nucleic acid amplification of pathogen-specific nucleic acid (i.e., target nucleic acid) with Magnetic Resonance Imaging (MRI) or Nuclear Magnetic Resonance (NMR) detection. The methods of the present invention can utilize rapid sample preparation, in part because MRI/NMR detection, unlike more conventional nucleic acid detection methods, generates a measurable signal in the radiofrequency range that is unaffected by the optical properties of the sample measured. The methods of the invention have been permitted by governments in a number of countries throughout the world and employed to detect the COVID-19 using both clinical (e.g., hospital or clinic, used for patient diagnostics) and dedicated MRI equipment in saliva and swab samples, without the need for nucleic acid extraction and purification steps.

The methods of the present invention result from parallel processing steps that allows hundred thousand samples a day at single location using a single MRI system, The MRI (or NMR) need not be integrated with other devices and instruments used prepare the samples and conduct the assay. Moreover, MRI detection is rapid and thus can be performed quickly, subject to availability of the instrument. Tens of thousands or more sample assays can be measured in a matter of minutes. The methods of the present invention provide two orders of magnitude greater capacity for the same dollar investments made, compared to the conventional PCR methods used during the COVID-19 pandemic, saving between 80 to 95% in infrastructure and operational costs, while the assay costs are of similar or lower cost.

This methods described herein enable rapid, sensitive, large scale detection of amplified nucleic acid samples corresponding large number of individuals screened. In one embodiment, LAMP assays are performed followed by MRI and/or NMR detection. Commercially available NMR systems and a wide variety of MRI imagers are used to detect target nucleic acids with high sensitivity and specificity.

The detection methods of the invention are also applicable to and adopted for detecting amplified nucleic acids produced by most known nucleic acid amplification methods, including PCR, RT-PCR, RAMP, RT-RAMP and any other method available today that uses primer sequences that can be modified with biotin (or another binding moiety).

The methods of the invention require complementary binding molecules, such as biotin in combination with the complementary binding molecule, streptavidin. Other complementary binding chemistry can be used in place of the biotin-streptavidin, provided it can be conjugated to primers.

MRI and NMR detection techniques of the invention utilize nanometer-scale paramagnetic particles (nanoparticles), which have been used clinically as MRI contrast agents and as research tools in vitro.

In one embodiment, nucleic acid amplification is accomplished using RT-LAMP as described below. When another amplification method is used, changes in the method may be needed. However, the necessary modification will be well within the level of ordinarily skilled artisan, given the teachings of the present disclosure.

The method of the invention has the following steps:
a. Providing a sample containing a target nucleic acid, e.g., a virus such as SARS-CoV-2. In certain embodiments, a LAMP means for collecting the clinical sample, is provided, such as a collection device (such as a tube and funnel for saliva, a cotton swab and tub to collect a respiratory sample).
b. Transporting the sample to a laboratory under conditions that preserve nucleic acids and prevent degradation thereof. The skilled artisan will appreciate that maintenance of suitable temperature and treatment with nuclease inhibitors may be required, depending on the amount of time between sample collection and processing in a laboratory.
c. Treating the sample to release the target nucleic acid, which can include heating the sample in the presence of reagents that lyse cells and/or dissociate remove nucleic acid-associated proteins, such as chelating agents (Chelex), proteinase K, guanidium hydrochloride, guanidium compositions and buffers.
d. Amplifying a region of the target nucleic acid in the presence of target-specific primers. In certain embodiments the nucleic acid is first reverse-transcribed using reverse transcriptase, and the resulting cDNA is amplified according to a LAMP or PCR protocol in the presence of a suitable DNA polymerase, nucleotides, co-factors and other components well known in the art. When LAMP is used, amplification is accomplished using a strand-displacing DNA polymerase, such as Bst polymerase, which has 5′→3′ DNA polymerase activity while lacking 5′→3′ exonuclease activity. When PCR is used, a thermostable DNA polymerase, such as Taq polymerase is used.
e. In certain aspects, one or more of the primers is biotinylated. Biotinylation can be accomplished by any method known the art including synthesizing primers with one or more biotinylated nucleotide(s), or chemically or enzymatically modifying the primer(s) following synthesis.
f. The conditions of time and temperature will vary depending on the method (e.g., PCR or LAMP) used to amplify the target nucleic acid. For example, isothermal LAMP is carried out at a single temperature while the temperature cycles between about 50° C. and about 100° C.
g. Contacting the amplified target nucleic acid with nanoparticles, wherein the nanoparticles specifically bind to the amplified target nucleic acid. To form target nucleic acid-nanoparticle complexes. In some embodiments, the nanoparticles are conjugated to streptavidin.
h. Exposing the target nucleic acid-nanoparticle complexes to a magnetic field device, which allows signal amplification in the presence of the target DNA.
i. Detecting the signal in an MRI system, which can identify the presence or absence of specific amplified target nucleic acid-nanoparticle complexes.
j. Collecting and analyzing the results of the MRI detecting using MRI image processing software.

In one embodiment, amplification relies on Reverse Transcription Loop Mediated Isothermal amplification (RT-LAMP) of nucleic acid to amplify COVID-19 RNA in a single-step, enabling rapid, ultra-high throughput, open access, or a random-access solution with semi-automated or automated processing platforms, and uses a commercially available MRI imagers to detect these assay product in very large numbers.

### Random Access Capability of Methods of the Invention

The present invention system and method permits integration of various sample preparation methods, various nucleic acid amplification methods, various assay types (such as LAMP assays, RT-LAMP assays, PCR, Reverse Transcription PCR (RT-PCR), Immuno-assays, ELISA, and the like), by making changes to the hardware and more importantly to the reagents, such as primers, nanoparticles and various probes that are compatible with magnetic processing and MRI or NMR detection. In one aspect of the the invention it is necessary to assemble random access compatible subsystems to process the human clinical samples from sample collection to RNA and DNA extraction from pathogen cells prior to RNA or DNA amplification and magnetic processing steps and prior to MRI detection.

According to certain aspects, the MRI-detected LAMP or RT-LAMP assay is used to detect nucleic acids from any pathogen, including viruses, bacteria and fungi) from any sample type such as saliva, swab, blood, urine, stool, sputum, stool and any fluid collected from a human body. The sample preparation method will be different for different sample types but the methods of the invention are substantially the same from LAMP amplification to MRI detection. This capability is also identified with the term “random sample access” or “random access, allowing various samples types to be processed differently in part, while keeping most or all of remaining system elements, and components unchanged.

Notably, the present invention avoids most of the bottlenecks above. For example, low-cost widely available machines and simple heaters are used to perform endpoint amplification (such as RT-LAMP and end point RT-PCR) which thereby imparts high throughput, open access, or random-access semi-automated or automated platforms, that also uses commercially available MRI systems for detection. In the steps of the present invention described above, there is a flexibility to vary each or any step without varying other steps. This random or open access capability of different elements of the inventive systema and MRI detection method helps to increasing volume of samples that can be analyzed in a short time.

For example, a sample collection method change from saliva to swab, does not affect other steps or the system components and hence can be changed. The random access capability also allows rapid change in detection of emerging pathogens, various clinical and environmental samples and their collection methods, different sample preparation methods, different types of LAMP and PCR primers and probes.

While performing large number of RT-LAMP assays detectable by MRI system, on thousands of SARS-CoV-2 virus specimens using 96 and 384 well heating blocks, 96 or 384 well sonication blocks, semi-automated or automated pipettes, or robots can dispense reagents and other liquids into the 9 plates for virus lysis and amplification, and detection. These steps can be performed simultaneously without the need for waiting for other steps to complete.

Employing random sample access or open access capability of different elements of MRI detection platform, namely sample collection, sample preparation followed by loop-mediated isothermal amplification (RT-LAMP) methods used for virus RNA prior to MRI detection streamlines the process and maximizes efficiency. LAMP methods can rapidly amplify a specific nucleic acid with high specificity under isothermal conditions with the use of four to six specifically designed, target specific primers. The LAMP reaction process requires no denaturation step, which is different from polymerase chain reaction (PCR), and DNA amplification occurs by means of the strand displacement activity of a DNA polymerase. The LAMP method has been widely applied for the detection of various microbes and pathogens including bacteria, and viruses. When detecting the RNA genome of a pathogen such as an RNA virus, LAMP has been combined with reverse transcription (RT) to provide an RT-LAMP assay of nucleic acid amplification.

The random access capability of the end-point detection of LAMP assays using MRI, allows detection of various types of pathogens, biomarkers, from clinical and environmental samples, all types of sample collection methods, various sample preparation methods and commercial kits, and different types of primers and probes, due to the highly flexible random access modality. For performing large number of RT-LAMP assays detectable by MRI for thousands of SARS-CoV-19 virus samples, it is possible to use industry standard 96- and 384-well heating blocks, 96- and 384-well sonication blocks, automated pipettes and robots that can dispense reagents and other liquids into the 96- or 384-well plates for viral lysis and RT-LAMP RNA amplification.

### Three Dimensional Simultaneous Detection of Assays With 3-D Sensor

Simultaneous detection of nucleic acid amplicons produced using RT-LAMP, LAMP, PCR or RT-PCR in the corresponding reaction tubes/wells, using MRI detection allows quantitative detection of all samples simultaneously when placed in the MRI sample coil. The MRI is an imaging device for visualizing small segments of the brain and/or body, and for qualitatively identifying the presence and/or absence of damaged cells or tissues, as well as abnormal structures such as tumors. The same concept is applied to in-vitro detection of large number of amplicons of PCR and LAMP assays. The image from a single to hundreds or even thousands of sample plates placed inside the MRI sample coil (head or body coils or whole body coils) gives a 3-D image after image processing, of all the samples placed as a three-dimensional array. This is analogous to the identification of different slices of human brain with MRI in the form of pixels or voxels. These pixels in 2-Dimension and Voxels in 3-dimension shows the properties of the brain corresponding to those pixels or voxels. The relaxation parameters measured in those specific areas of the brain tissue are reconstructed as a 3-D image and its 2-D slice. The pixels plotted in two dimensions of a three dimensional array of assays is used to visualize the results and convert them in the positives and negatives represented by 1 and 0.

The methods of the invention can detect three dimensional arrays of samples, unlike the conventional optical detection systems. While each assay is amplified and detected using an optical detector, the light from the sensor is shined on each assay using a dedicated light source after every few amplification cycles or times. Unlike the prior art methods the methods disclosed herein use DC and AC magnetic fields and sequences of DC and RF pulses that allows the detection of the signal and the identification of the sample position or number. Prior art methods use light sources and optical detection systems that must be present right next to the sample, making this system highly integrated to the system as a whole, and hence the system is fully dedicated this purpose under processing from start-to-end of the detection process.

The methods of the invention employing MRI technology allows the detection of very small volumes in three dimensions and allows processing of the signal from each small volume in the three-dimensional space. MRI has been used for in-vivo medical imaging for decades, while using the same methods for pandemic screening has not been previously described. The invention provides detecting tens of thousands of samples in 3-D format is facilitated by the integration of various modern developments in science and technology such as Genome science, nanotechnology, synthetic gene segments, functionalization of gene sequences, binding chemistry for performing rapid assays, magnetic field enabled assay processes, rapid sample collection methods, rapid sample lysis and digestion methods for human or animal specimens, functionalized nano particles, image processing methods and software tools and also the commercially available MRI systems and the expertise in the MRI area.

The present invention provides methods for MRI detection of nucleic acid amplicons that is radically different from the current state of the art because of the 3-Dimensional detection system that can identify each sample in a 3-D stack of samples placed one on top of another in 3-D array pattern. The radiofrequency waves and magnetic fields act synergistically to identify each sample using magnetic field gradients and radiofrequency phase encoding methods. These magnetic fields and their properties identify the location of each sample with an image, that has the signature of the sample present, using a pixel in the MRI. The pixels in the MRI image collected from each sample tube identifies the presence or absence of specific nucleic acid sequences.

Using the methods of the invention, almost every MRI system available in the healthcare system could be used “as-is” or easily adapted for pathogens detection in the human or animal samples. The MRI systems with magnetic fields over a wide range, have been validated using this method, and thereby allowing global deployment of these systems to fight the spread of the COVID-19 disease all over the world. MRI systems with low magnetic fields (between 65 mill Tesla to 3000 milli Tesla) have been tested and give same substantially the same results in identifying the presence or absence of pathogens in clinical samples.

MRI-detected nucleic acids give the same result irrespective of the magnetic field, as demonstrated by comparison of samples detected on many MRI systems, namely: low field commercial MRI systems Hyperfine MRI with 65 Milli Tesla field, the Esaote C-Scan with 200 milli Tesla field, Esaote 0-Scan with 300 milli Tesla and intermediate field ONI MRI system with 1000 milli Tesla field, Siemens Magnetom system with 1500 milli Tesla field and 3 Tesla Varian and GE systems. In addition, the methods of the present invention have been verified on wide range of MRI systems from various vendors and systems installed all over the world.

The use three-dimensional visualization capability on small amounts of assay liquids, using MRI allows visualization of data processing of large numbers of samples simultaneously that are processed on an hourly basis or in minutes. There is no detection bottleneck in the proposed MRI detection system as there is no sub-system that can slow down other steps, that does not happen in a sequential manner.

Unlike prior art methods, the present MRI method is not connected to any sample preparation system, and does not require pure samples as the radio frequency signal is not highly affected by the purity of the sample. Unlike the optically-detected real-time PCR and RT-LAMP systems that take very long times to prepare and process, due to removal from nucleic acid samples of inhibitors that can interfere with the PCR and LAMP amplification at the same time requires the removal inhibitors for optical signal detection. The methods of the present invention are not affected by the optical inhibitors and hence rapid lysis methods and sample processing methods using chemical processing or heating of the sample is all that is needed.

The MRI detection methods of the invention partly rely on prior art of the magnetic resonance and magnetic detection techniques, involving nanometer scale paramagnetic particles (nanoparticles) used in-vivo as MRI contrast agents. In the prior art, these magnetic nanoparticles are injected into a patient or animal prior to MRI imaging. When the contrast agents selectively bind to the target cells they cause a local change in the spin relaxation parameters leading to contrast variations in the MRI image thereby increasing the image contrast and visualization of e.g., tumor and non-tumor cells in the human body.

The present invention uses similar nanoparticles principles, but they are applied to nucleic acids that are difficult to detect due to the low concentrations present in clinical samples. The clinical samples that can be tested using nanoparticle-mediated MRI and NMR detection include non-invasively collected saliva, nasal swab, sputum, urine and stool samples as well as more invasively collected samples such as blood and tissue.

Quantification of Single Tests with NMR In One-Dimension:

In another aspect of the invention, using the same basic method known as Nuclear Magnetic Resonance (NMR), which is the fundamental technology behind MRI, processed assays are detected using commercial NMR systems to further identify, verify or to quantify the viral or bacterial pathogen loads, and their strains in the individual specimens in one-dimension. In contrast, the embodiment of using MRI for high-throughput analysis of large numbers of the samples without quantification is defined as detection in three-dimensions. This one-dimensional detection also relies on the RT-LAMP and RT-PCR assay elements such as known primers (synthetic nucleic acid segments) for each strain of the same pathogen or different pathogens altogether. The MRI systems used for detection are the in the NMR frequency range of 2.5 mHz to 150 MHz. NMR systems are used due to the lower and intermediate end of the magnetic field strengths that can have less influence on the magnetic nanoparticles used as the detection probes.

These preferred NMR systems include homemade and commercial benchtop NMR systems including Bruker minispec systems, Thermo Fisher Pico Spin system, Magritek bench top NMR systems, Waveguide formula handheld NMR system, and a wide range of Bruker and Varian NMR machines in the frequency range between 2.5 MHz and 150 mHz.

### MRI Detected LAMP and RT-LAMP Assay Design:

Certain methods of the present invention approach detecting amplified DNA, using LAMP or RT-LAMP assays, that require six different primers namely F3, B3, FIP, BIP, Loop F and Loop

The LAMP is characterized by the use of first four primers specifically designed to recognize 6 distinct regions of a target gene. The LAMP assay is well known and is illustrated diagramatically on the world wide web, for example, at www [dot] neb [dot]com/applications/dna-amplification-per-and-qper/isothermal-amplification/loop-mediated-isothermal-amplification-lamp

The four primers used are as follows:

a. Forward Inner Primer (FIP): The FIP consists of a F2 region at the 3′end and a F1c region at the 5′end. The F2 region is complementary to the F2c region of the template sequence. The F1c region is identical to the F1c region of the template sequence.
b. Backward Inner Primer (BIP): The BIP consists of a B2 region at the 3′end and a B1c region at the 5′end. The B2 region is complementary to the B2c region of the template sequence. The B1c region is identical to the B1c region of the template sequence.
c. Forward Outer Primer (F3): This primer consists of a F3 region which is complementary to the F3c region of the template sequence. This primer is shorter in length and lower in concentration than FIP.
d. Backward Outer Primer (B3 Primer): This primer consists of a B3 region which is complementary to the B3c region of the template sequence. After the important LAMP primer set (FIP, BIP, F3, and B3) has been determined, the loop primers, which reduce the amplification time and improve the specificity, are designed.

The step-by-step approach used in the prior art of LAMP or RT-LAMP are described in the literature and prior art and briefly described below to make the connection with the inventive MRI detected method and to highlight the changes needed in implementing the new inventive method and also to understand the elements of the MRI detected LAMP products.

In the Prior art of LAMP method, RNA in the sample solution and the components of the reaction solution with MRI detectable biotinylated primers described earlier, are incubated at a constant temperature between 60-65° C. As a result, the following steps were observed. The process is detailed below with reference to the Figure illustrating “RT-LAMP in RT-LAMP method—Principle: RT-LAMP” viewed on the world wide web at loopamp [dot]eiken [dot]co [dot]jp/e/lamp/rtprinciple.html

RT-LAMP STEP-1: The BIP primer anneals to the template RNA, and with the activity of reverse transcriptase, cDNA is synthesized. Uniquely Biotinylated primers are used in the amplification using the sixteen possible combinations of the primers that have biotin at the 5′ end. A commercial vendor (IDT Technologies, USA) provided these, supplied with the biotin location and design. These primers were selectively biotinylated in order to bind to nanoparticles that are important for the post processing of RT-LAMP products. Any of the embodiments that use biotin-conjugated primers can participate in the LAMP or RT-LAMP assay and be detected downstream using MRI method or NMR method.

The BIP or FIP primers initiate strand-displacement as a result of their B2 and F2 portions binding to complementary sections of the target sequence. This process occurs by displacement by, e.g., Bst polymerase in the reaction medium.

RT-LAMP STEP-2: The F3 primer anneals to the region outside of the BIP primer; with the activity of reverse transcriptase, a new cDNA is synthesized, while concurrently releasing the cDNA strand previously formed by the BIP primer.

RT-LAMP STEP-3: From step (2), the single-stranded cDNA synthesized from BIP is released. The FIP primer then anneals to this single stranded cDNA.

RT-LAMP STEP-4: From reverse transcription step (3), through the activity of DNA polymerase with strand displacement activity, the 3′ end of F2 region in FIP becomes the starting point to synthesize complementary DNA strand.

RT-LAMP STEP-5: The F3 primer anneals to the region outside of FIP, and its 3′ end becomes the starting point to synthesize while concurrently releasing the DNA strand previously formed by FIP.

RT-LAMP STEP-6: The DNA strand synthesized by F3 primer together with the template DNA strand forms a double stranded DNA.

RT-LAMP STEP-7: Since the FIP-linked DNA strand, which was released in step (5), contains complementary sequences at both ends, it self-anneals and forms a dumbbell-like structure. This structure (7) becomes the starting structure of the LAMP cycling amplification.

RT-LAMP STEP-8: The dumbbell-like DNA structure (7) is quickly converted into a stem-loop DNA by self-primed DNA synthesis, which unfolds the loop at 5′ end to extend DNA synthesis. The BIP anneals to the single stranded region in the stem-loop DNA to start DNA synthesis in step (8) while releasing the previously synthesized strand.

RT-LAMP STEP-9: This released single strand forms a stem-loop structure at the 3′ end because of complementary F1c and F1 regions. Then, starting from the 3′ end of the F1 region, DNA synthesis starts using the self-structure as a template, and releases the BIP-linked complementary strand. Structure (9) is formed.

RT-LAMP STEP-10: The released BIP-linked single-strand then forms a dumbbell-like structure as both ends have complementary F1-F1c and B1c-B1 regions, respectively. This structure is the turn-over structure of structure (7).

RT-LAMP STEP-11: Similar to step (7), structure (10) proceeds self-primed DNA synthesis starting from the 3′ end of the F1 region. Furthermore, the FIP anneals to the F2c region and starts synthesizing DNA strand. This FIP-linked DNA strand is released by the strand displacement of self-primed DNA synthesis. Accordingly, similar to step (7), (8) and (10), step (10) and (11) proceeds, and structure (7) is once again being formed.

RT-LAMP STEP-12: With the structure produced in step (9) (or step (12)), the FIP (or BIP) anneals to the single stranded F2c region (or B2c region), and DNA synthesis continues by releasing double stranded DNA. As a result of this process, various sized structures consisting alternately inverted repeats of the target sequence on the same strand are formed.

In all the above steps, the biotin is preserved and available in the products as well as in the leftover products that are utilized in the downstream preparation of the assay for MRI detection using MRI imaging.

When using MRI method, the successful LAMP assays after successful amplification results in “darker spots” in the MRI image representing shorter MR relaxation times called T2, compared with the unsuccessful reactions giving “bright spots” in the MRI image representing longer MR relaxation times called T2. The switching of darker to brighter and vice-versa is also possible with the selection of nanobeads that have the capacity to change the relaxation times due to their interactions with functional groups in the presence of magnetic fields.

In one embodiment of the invention, the LAMP primers that are involved in amplifying DNA are biotinylated as shown below. The complementary functional group, Streptavidin, is attached to nanoparticles that are added to the assay, which are an essential element of the Magnetic Resonance-based detection process especially in the case of MRI detection in three dimensions.

In the an embodiment of the invention using LAMP, biotin is attached to the primers BIP, FIP, Loop F and Loop B at the 5′-end of the primers. Shown below are the several different permutations and combinations of primers, that makes the MRI detection possible.

### 1. 5′-F3-3′

### 2. 5′-B3-3′

### 3. 5′-Biotin-FIP-3′

### 4. 5′-Biotin-BIP-3′

### 5. 5′-Biotin-Loop F-3′

### 6. 5′-Biotin-Loop B-3′

Biotinylation of F3 and B3 does not give successful signals or differences between positive and negative LAMP assays and hence F3 and B3 are not biotinylated. The 5 other possible binding options from 3 to 6 and all permutations and combinations give MRI images that show different positive and negative assay intensities.

In the six LAMP primers listed above, primers F3 and B3 will not have the functional element such as Biotin at 5′ end. These two primers when added with functional groups attached with them fail to differentiate the presence and absence of the target DNA and hence not considered in inventive MRI detected LAMP or RT-LAMP assays. In the six LAMP primers listed above, at least one of the four primers FIP, BIP, Loop F and Loop B are biotinylated as the functional group where streptavidin is the complementary functional group required to be attached or coated on the nanobeads that alters the MRI properties leading to the differentiation of positives and negatives.

The preferred combinations of LAMP primers that are added with biotin group at the 5 ‘-end (that is also called biotinylated at 5’-end of the primer) are listed below in the order they are preferred in the inventive method.

Biotin at 5′ end of FIP primer only,
Biotin at 5′ end of BIP primer only,
Biotin at 5′ ends of FIP and BIP primers,
Biotin at 5′ end of Loop F primer only,
Biotin at 5′ end of Loop B primer only,
Biotin at 5′ ends of Loop F and Loop B primers,
Biotin at 5′ ends of FIP primer and Loop F primer,
Biotin at 5′ ends of FIP primer and Loop B primer,
Biotin at 5′ ends of BIP primer and Loop F primer,
Biotin at 5′ ends of BIP primer and Loop B primer,
Biotin at 5′ ends of FIP Primer, BIP primer and Loop F primer,
Biotin at 5′ ends of FIP Primer, BIP primer and Loop B primer,
Biotin at 5′ ends of FIP Primer, Loop F primer and Loop B primer,
Biotin at 5′ ends of BIP Primer, Loop F primer and Loop B primer,
Biotin at 5′ ends of FIB primer, BIP Primer, Loop B primer and Loop F primer.

In one embodiment of MRI the detected LAMP assay, Biotin has been attached to 5′-end of the FIP LAMP primer only, making it the lowest cost primer. In the simplest design no other primer is biotinylated. Alternatively, the BIP primer can be biotinylated leaving FIP unfunctionalized. In the simplest design, one of the two from FIP and BIP LAMP primers can be the one of two primers that is modified in the LAMP reaction and hence this less expensive in terms of primer cost as the manufacturers charge significant money for each primer Biotinylated. The matching magnetic nanoparticle is coated with streptavidin so that the “un-used” biotinylated FIP primers will strongly bind to streptavidin attached with the magnetic nanoparticle allowing easy scavenging of the nanoparticles in the absence of specific LAMP reaction.

In one embodiment, Biotin has been attached to 5′-end of the BIP LAMP primer. The FIP primer is not biotinylated making this design very simple. The BIP LAMP primers is the only primer that needed to be modified in the LAMP reaction. The matching magnetic nanoparticle is coated with streptavidin so that the “un-used” biotinylated BIP primers will strongly bind to streptavidin in the magnetic nanoparticle allowing easy scavenging of the nanoparticles in the absence of specific LAMP reaction.

In one embodiment Biotin has been attached to 5′-ends of both FIP and BIP LAMP primers. Making modifications to two primers FIP and BIP LAMP primers is more expensive in terms of primer cost as the manufacturers charge significant money for each primer attached with binding molecules such as Biotin. The matching magnetic nanoparticle is coated with streptavidin so that the “un-used” biotinylated FIP and BIP primers strongly bind to streptavidin in the magnetic nanoparticle allowing strong affinity and easy scavenging of the nanoparticles by both FIP and BIP primers in the absence of specific LAMP reaction.

In one embodiment, Biotin has been attached to 5′-end of the BIP LAMP primer. The FIP primer is not biotinylated, making this design very simple. The BIP LAMP primers is the only primer that needed to be modified in the LAMP reaction. The matching magnetic nanoparticle is coated with streptavidin so that the “un-used” BIP primers attached with Biotin will strongly bind to streptavidin in the magnetic nanoparticle allowing easy scavenging of the nanoparticles in the absence of specific LAMP reaction.

In one embodiment, Biotin has been attached to 5′-ends of both FIP and BIP LAMP primers. Making modifications to two primers FIP and BIP LAMP primers is more expensive in terms of primer cost as the manufacturers charge significant money for each primer attached with binding molecules such as Biotin. The matching magnetic nanoparticle is coated with streptavidin so that the “un-used” FIP as well as BIP primers attached to Biotin will strongly bind to streptavidin in the magnetic nanoparticle allowing strong affinity and easy scavenging of the nanoparticles by both FIP and BIP primers in the absence of specific LAMP reaction.

In one embodiment of the invention the Streptavidin conjugated Super Mag nanoparticles of 20 to 200 nanometers are used with maximum flexibility, speed and sensitivity. The size of the nanobeads, the binding capacity of the nanoparticles is determines the optimum concentration of the nanobeads in the assay.

The size of the nanoparticle used in the detection varies in the MRI detection. The smaller nanobeads are used in large magnetic field MRIs and large nanobeads are used in low-field MRI systems to make the detection process easier and sensitive.

In an embodiment of the invention, the Super Mag nanoparticles with monolayer of streptavidin that is covalently coupled to the surface of the nanoparticles makes most of the biotin binding sites sterically available for binding of biotinylated primers that are un-used and did not participate in the LAMP reaction. With large surface area, better colloidal stability and unique surface coating, Streptavidin Super Mag exhibit high binding capacity, low non-specific binding and fast magnetic binding for scavenging.

The MRI detection method described herein is an end-point detection approach that allows rapid, large-scale, extraction-free sample processing followed by MRI detection system. This is contrary to the prior art detection technologies detecting the presence of nucleic acid amplicons from DNA and RNA uses wide range of physical and chemical principles such as real-time quantitative polymerase chain reaction (rt-qPCR), Reverse Transcription quantitative polymerase chain reaction (RT-qPCR) detected that are mostly detected fluorescence spectroscopy using the dedicate sensor watching the sample. In the case of isothermal amplification methods LAMP or RT-LAMP the amplicons are mostly detected using turbidity, calorimetry, fluorescence less frequently in the end-point and mostly in real-time detection mode.

Many applications of the invention were tested, validated and adopted using similar approaches to those explained above, for different gene segments of the COVID-19 virus RNA and also for other viruses Influenza A and B virus RNA segments, many other viruses, various strains of the COVID-19 and other virus variants that are emerging and also various gene segments that are mutating.

The most important element of the inventive method is making any RT-LAMP or LAMP assay detected by any other detection method, into a MRI detectable 3-D format. In certain embodiments of the invention the unique combination of selective LAMP primers designed and attached to specific binding molecules that are used to bind to the magnetic nanoparticles coated with the matching binding moieties with binding molecules on those specific LAMP primers.

The MRI signal creation occurs due to the binding of nanoparticles attached to the biotin moieties present in various primers and amplification products, which are the essential elements of this method that lead to the variation of relaxation parameters namely T2 and T1 that gives the contrast variation together, where are T2 being the most important parameter both in MRI imaging known as T2 weighted imaging and in NMR the detection and quantification is done using a T2 measurement. The 3-D MRI detected image intensity has variations that are generated by these magnetic nanoparticles in the presence and the absence of specific target nucleic acids such as the target RNA and DNA, and also by the presence of the specific LAMP primers that are unused in the reaction tube.

At the end of a successful LAMP reaction the specific target LAMP primers are mostly consumed by the amplification process in the presence of target nucleic acids that are amplified by the reaction. In the absence of specific target nucleic acids the primers are leftover in the reaction tube. These elements of the reaction allows unique identification of these reaction products, that results in varying signal intensities of MRI signal.

In the case of specific LAMP amplification, specific LAMP primers with binding molecules are shielded by the successful LAMP reaction process. In the successful LAMP reaction the nanoparticles added later to the reaction mix will not find the binding molecules that are “shielded” or ‘hidden” by the specific LAMP amplification step. In the absence of LAMP specific reaction the primers will be freely floating, because the reaction has all the primers that are ‘leftover’ that has binding molecules effectively bind to the functionalized nanoparticles. The binding of “leftover” primers via binding molecules with nanoparticle probe molecules, that are coated with a complementary binding chemistry, forming strong binding that cannot be separated during the reaction.

Thus a successful LAMP reaction, that results in the “consumption of primers”, deprives the primer binding sites for nanoparticles and hence the nanoparticles stay dispersed and any weak association between nanoparticles are easily disrupted under mechanical forces. Thus, a successful specific LAMP reaction makes the magnetic nanoparticles uniformly distributed in the LAMP reaction tube. In addition, the large amount of specific LAMP products, leads to further dispersion of the magnetic nanoparticles in the assay. Thus, an unsuccessful LAMP reaction leads to uniform distribution of magnetic nanoparticles in the reaction medium leading to a predetermined nuclear spin relaxation, in the event of specific LAMP reaction.

Alternatively, the binding of magnetic nanoparticles to the “un-used” primers leads to an effective scavenging of nanoparticles by these primers that enables magnetic separation and non-uniform distribution of magnetic nanoparticles. When there is a strong binding between the specific “unused” primers and nanoparticles, the magnetic nanoparticles cannot be easily dispersed by mechanical forces and hence results in non-uniform distribution of magnetic nanoparticles in the assay medium results in unique relaxation times leading to significantly different signals that makes the LAMP assays highly reliable, rapid and largescale.

When using MRI, the successful LAMP assays after specific amplification results in “black holes” or “darker spots” or shorter relaxation times, compared with the non-specific or absence of the products which results in “bright spots” with longer relaxation times. In another embodiment the opposite result has been demonstrated by chemically changing the surface properties of the nanoparticles binding with biotin present in the RT-LAMP primers and products. That means the “darker spots” represent the absence of virus RNA or and “brighter spots” represent the presence of virus RNA. This is opposite signal was achieved by the addition of chemicals that can alter the surface properties in the assay medium prior to RT-LAMP amplification step.

### MRI Detected PCR or RT-PCR Assays

In another embodiment of the invention, RT-PCR amplicons are synthesized for detection using MRI with specific design of primers. The RT-PCR requires two primers namely Forward and Reverse primers that can also target the gene segment of COVID-19 that is also detected by RT-LAMP. The amplicons from RT-PCR and RT-LAMP are qualitatively very different. The RT-PCR primers are also Biotinylated at the 5′-ends of both the PCR primers.

### More Systems and Subsystems for MRI Detectable Assays

One or more of the following processes, methods and sub-systems are associated with the inventive method for rapid and sensitive detection of nucleic acids using LAMP or RT-LAMP assay.

The mixed product undergoes unique process in the presence of a magnetic array of large number of magnets that promotes the reaction to happen faster and this process increases the signal dramatically by magnetically enhancing the nucicid signal and more precisely this process increases the contrast, or strength of signal, or the signal difference between negatives and positive assays.

The MRI image is collected using a clinical MRI device using the array of all the processed samples to see them visually or/and later using software program that will read, transfer and process the MRI data from 3-D into linear data showing positives and negatives very fast.

The MRI images are processed using available image processing software tools such as Fiji (ImageJ) and Matlab programs developed for this purpose to declare the results of the test automatically using the software that can be used with all the MRI machines available for measuring signal from the reaction tubes.

### EXAMPLES

### Example—1

Detailed description of the steps of the preferred embodiment and commercially sold assays for COVID-19 pandemic screening that has received approvals from various Governments in the world including in the Americas, Africa, Asia and elsewhere and used worldwide to detect COVID-19 specimens in the form of Saliva.

### Specimen Collection:

Saliva and swab specimens were collected in many locations in India, Nigeria and USA and comparative tests were done with Gold Standard RT-PCR test method, to show that the detection using MRI of RT-LAMP and RT-PCR are close 95 to 100 percent and the results are comparable with standardized tests.

More than 1,000 human saliva and nasal swab samples were collected from hospital visitors to an India hospital and medical college (SVS Medical College, Mehbubnagar, India) and these samples were tested with multiple methods to show the correlation between the results. The RT-qPCR assay kits were purchased from Thermo Fisher and known as TaqPath™ COVID-19 Combo Kit. The real-time quantitative RT-PCR test performed on RNA extracted from the swab and saliva samples, using Qiagen RNA Extraction Kits. The commercial system employing Prior Art to perform RT-qPCR assays is called Quant Studio 5 is also purchased Thermo Fisher, USA.

The number of saliva samples tested and compared in India were 250, in which 119 samples were negatives for COVID-19 virus and 131 were positive saliva samples. The comparison study using RT-qPCR detected using Quant Studio (Fluorescence based optical detection), and RT-LAMP is detected using MRI, NMR and Gel Electrophoresis. The same saliva samples and the swab samples from the same patients were also tested using all these methods and the results showed near perfect match between the technologies showing 97% match in positive samples and 99% match in negative samples.

The widely used Molecular tests (RT-PCR and RT-LAMP) for COVID-19 were performed from the beginning on nasal swab samples. The collection of nasal swabs from 2 to 3 million people every day required hundreds of thousands of healthcare workers and nurses. One of the bottlenecks in increasing the testing volume to tens of millions of people was removed by saliva specimen collection. The method used self-collected saliva. The saliva collection, is a tube done using a low-cost device that consists of a plastic funnel and 5 mL tube that is screw connected with funnel. The person self-collecting saliva will replace the funnel with a screw cap after filling the saliva. The saliva volume needed to perform the inventive method is less than 10 microliters. The amount of saliva collected was anywhere between 100 uL to 1 mL. After collection of saliva into the collection tube or vial, the saliva is transported to the testing facility that is a stationary or mobile lab. The saliva is kept in temperature around 4 to 8° C. The saliva sample was kept in temperature between 20 and 30° C. for one to two days without negatively affecting the quality and the initial quantity of the COVID-19 virus into the vial. As another option, the method also processed nasal swab specimens. The swab samples stored in viral transport medium (VTM) were processed directly following the same process adopted for Saliva. The amount of saliva and swab VTM processed is around 4 to 6 uL in a RT-LAMP reaction of 25 uL.

b. COVID-19 Test Specimen Packing and Shipping

The saliva samples were transported to the test location within the same day and there was no need for any preservation solution. The shipping of samples in a secured decontaminated package is send via mail or directly delivered by the collection staff.

c. The COVID-19 Virus present in the saliva was inactivated and lysed using a simple process in the inventive method. The make it safe and easy to add into the 250 uL RT-LAMP reaction tubes, hot water at the temperature between 85 to 98 degree C. was added. The amount of water added is approximately equal to the amount of saliva collected. This inactivation also kills the virus and in order to release the RNA from the virus, the lysis step is performed using saliva processing buffer and chemicals. The addition of lysis buffer is followed by heating of the mix of saliva processing buffers and chemicals at temperatures between 90 and 98 degree C. for 2 to 10 minutes. Higher the hearing temperature lower the heating time. In the preferred embodiment 95% degree C. for 10 minutes was applied. In the preferred embodiment of the inventive method a chaotrope agent and strongest denaturants Guanidinium salts are used in the form of liquid in a concentration range of 10 uM to 100 uM, to reduce the activity of saliva enzymes, to increase the solubility and to reduce the viscosity of the of saliva.

d. The RT-LAMP reaction was performed using RT-LAMP master mix readily available at lease three vendors Lucigen, USA New England Bio Labs (NEB), USA and Optigene, UK. The commercial ready to use master mix with CatLog numbers 1700 and 1804 were used. The primer design for RT-LAMP for COVID detection using MRI method was performed using several sets of RT-LAMP primers. Each primer set targeted a specific region of the COVID-19 genome. The primers available in the published literature were used. As described in the previous section of the patent, all the permutations and combinations of biotin addition were verified using the following list of primers.

Several LAMP primers from the literature were validated and in some cases new primers were designed and optimized (for different COVID-19 gene segments, variants and other targets). The primer set listed below for the preferred embodiment of MRI detected RT-LAMP is obtained from the publication (Reference 1.) and the modifications were made to the primers to allow MRI detection that is primarily enabled by biotin addition and optional purification was performed HPLC purification process offered by the commercial vendor (Integrated DNA technologies (IDT), USA).

Reference—1: Rabe, B. A. & Cepko, C. SARS-CoV-2 detection using isothermal amplification and a rapid, inexpensive protocol for sample inactivation and purification. Proc. Nat. Acad. Sci. (PNAS) 117, 24450-24458 (2020).

### Primer Selection and Design of Synthetic DNA

The biotinylated oligos for Orflab gene segment namely F3, B3, Biotin-FIP, and Biotin-BIP were designed modifying the primers available in the reference above and a set of loop primers (LF and LB with and without Biotin at the 5′-end) were designed by filling the gap of the gene segment chosen by filling the gaps and a linker. The synthetic DNA was made using the same software and synthesized with the help of commercial vendor (IDT, USA)

All oligos were ordered from IDT and resuspended in TE provided by IDT, USA water at a 100 μM concentration. Oligos were combined to make 100 μL of 10× primer mix as follows: 4 μL of Biotin-FIP, 4 μL of Biotin-BIP, 0.5 μL of F3, 0.5 μL of B3, 1 μL of Biotin-LF, and 1 μL of Biotin-LB, and brought to 100 μL with nuclease free water. All RT-LAMP reactions were set up as described by NEB protocols (E1700 and M1804) and run at 62° C.

All viral RNA sequences used in this study were purified RNA and number of different virus controls received from BEI Resources for different COVID-19 strains diluted serially in nuclease free water.

The preferred embodiments of the MRI detected RT-LAMP primer combinations are validated and presented using the following primer-biotin combinations. The primers are deigned with biotin attached as per the above listed 18 combinations described above and the MRI images was recorded and the images showed the presence and absence of COVID-19 gene segment that was amplified.

The invention was verified for COVID-19 virus that has the RNA converted into cDNA which was amplified using LAMP method and also using killed COVID-19 virus in the lab and later in the clinical labs with thousands of samples collected from COVID-19 virus infected people. The matching target COVID virus, matching target RNA and the matching cDNA resulted in darker image when MRI image was recorded after post processing the LAMP amplicons verifying the invention.

The synthetic cDNA designed and synthesized commercially with the help of IDT, USA is given below.

| ORF1ab gene target sequence(Nucleotides 2101-2581 of Genbank Accession | |
|---|---|
| No. NC_045512.2): | |
| (SEQ ID NO: 10) | |
| 2101 | gctaactaac atctttggca ctgtttatga aaaactcaaa cccgtccttg attggcttga |
| 2161 | agagaagttt aaggaaggtg tagagtttct tagagacggt tgggaaattg ttaaatttat |
| 2221 | ctcaacctgt gcttgtgaaa ttgtcggtgg acaaattgtc acctgtgcaa aggaaattaa |
| 2281 | ggagagtgtt cagacattct ttaagcttgt aaataaattt ttggctttgt gtgctgactc |
| 2341 | tatcattatt ggtggagcta aacttaaagc cttgaattta ggtgaaacat ttgtcacgca |
| 2401 | ctcaaaggga ttgtacagaa agtgtgttaa atecagagaa gaaactggcc tactcatgcc |
| 2461 | tctaaaagcc ccaaaagaaa ttatcttctt agagggagaa acacttccca cagaagtgtt |
| 2521 | aacagaggaa gttgtcttga aaactggtga tttacaacca ttagaacaac ctactagtga |

The six COVID-19 LAMP primers that target ORFlab gene for this preferred embodiment is shown below. The FIP and BIP primers were designed with Biotin molecule at their 5 ends.

F3 Primer:
(SEQ ID NO: 1)
5′-CGGTGGACAAATTGTCAC-3′
B3 Primer:
(SEQ ID NO: 2)
5′-CTTCTCTGGATTTAACACACTT-3′
FIP Primer:
(SEQ ID NO: 3)
5′-Biotin-TCAGCACACAAAGCCAAAAATTTATTTTTCTGTGCAA
AGGAAATTAAGGAG-3′
BIP Primer:
(SEQ ID NO: 4
5′-Biotin-TATTGGTGGAGCTAAACTTAAAGCCTTTTCTGTACAA
TCCCTTTGAGTG-3′
Loop F Primer:
(SEQ ID NO: 5)
5′-TTACAAGCTTAAAGAATGTCTGAACACT-3′
Loop B Primer:
(SEQ ID NO: 6)
5′-TTGAATTTAGGTGAAACATTTGTCACG-3′

In this embodiment of RT-LAMP primer sequence for COVID-19, both FIP and BIP primers were designed with attached Biotin molecule at their 5 ends.

The design of the same six LAMP primers that target the same ORF lab gene in other permutations and combinations are presented below: The BIP, FIP, Loop B and Loop F are attached with Biotin molecule at the 5′ end of the primers.

Below are the primers that has all the tested embodiments has at least one biotin in one or more of the four chosen primers (FIP, BIP, Loop B and Loop F) that show measurable and observable difference between positive and negative specimens. Having biotin in all 4 primers is less preferred though this embodiment also gives contrast difference between positive and negative samples there is no need to attach biotin in all four identified primers primarily because the cost is higher to add more biotin moieties in more primers, that makes no additional benefit while detecting the positives and negatives using MRI in 3-D which is a qualitative detection method and not quantitative method.

F3 Primer:
(SEQ ID NO: 1)
5′-CGGTGGACAAATTGTCAC-3′
B3 Primer:
(SEQ ID NO: 2)
5′-CTTCTCTGGATTTAACACACTT-3′
FIP Primer:
(SEQ ID NO: 3)
5′-Biotin-TCAGCACACAAAGCCAAAAATTTATTTTTCTGTGCAAA
GGAAATTAAGGAG-3′
BIP Primer:
(SEQ ID NO: 4)
5′-Biotin-TATTGGTGGAGCTAAACTTAAAGCCTTTTCTGTACAAT
CCCTTTGAGTG-3′
Loop F Primer:
(SEQ ID NO: 5)
Biotin-5′TTACAAGCTTAAAGAATGTCTGAACACT-3′
Loop B Primer:
(SEQ ID NO: 6)
5′-Biotin-TTGAATTTAGGTGAAACATTTGTCACG-3′

The sixteen embodiments that are derived from the biotin attached four primers are applicable to any LAMP and RT-LAMP assay which comprises six primers used in the assays.

In the preferred embodiment the above described primers with their biotin combinations are added to commercially available single-tube RT-LAMP master mix sold by New England Bio Labs (catalog numbers NEB 1700 series and/or NEB 1804 series) or OptiGene RT-LAMP master mix, or Lucigen RT-LAMP master mix. This mix with optimized primer mix of above 6 primers with various concentrations (4 to 16 uM of FIP and BIP, 1 to 4 uM of Loop B and Loop F primers, and 0.5 to 2.0 uM of F3 and B3 primers). This master mix is added to the the heat treated, buffer treated and homogenized saliva to perform RT-LAMP amplification of COVID-19 virus and other RNA viruses listed above. The amplification step involves, addition of enzymes and other components that are used to perform Reverse Transcription (RT). The amplification step also involves nucleic acid amplification using another enzyme present in the reaction reagents and buffers. The temperature between 60 and 67 degrees C. is applied to perform LAMP reaction. After the completion of RT-LAMP reaction that is performed between 60 and 65 degrees for NEB and OptiGene master mixes and 67 degree C. for Lucigen RT-LAMP master mix. The preferred assay employs same temperature to perform the steps RT and LAMP, that is performed at the same temperature. After amplification step that can last between 10 minutes to 60 minutes in general though in the preferred embodiment it is restricted to 15 and 30 minutes. The time is adjusted depending on the performance expectations parameters defined for the application. Once amplification is completed an optional step of heating the RT-LAMP assay reagents to 85 degree C. to truncate or stop any possible unplanned reaction after amplification is completed. And the amplified sample tray with 8, 96 or 384 samples are removed from the heaters and saved in the refrigeration temperature.

Though the steps a to d are sane for both the embodiments (RT-PCR and RT-LAMP) the step described above for RT-LAMP has equivalent but different elements or steps in making MRI detected RT-PCR amplicons. The RT-PCR primers targeting the gene segment of ORFlab gene is presented below with just one configuration of Biotin attached to both the primers.

The RT-PCR parameters used to detect MRI detected amplicons are the following. The bright images with long T2 represent positive samples and the darker spots with short T2 represents Negative sample. The PCR primers for RT-PCR test were synthesized by IDT, that are show below. Each PCR reaction mix contained 1× PrimeTime® Gene Expression Master Mix (purchased from IDT, USA), 0.25 uM concentration of PCR primers, and 10 fg of synthesized COVID-19 target shown (made by IDT, USA). The PCR was carried out with a Applied Biosystems Veriti 9902 4375786 PCR 96 Well Thermal Cycler with a temperature profile of 95° C. for 3 min, followed by 50 cycles of amplification (95° C. for 15 s and 60° C. for 1 min)

Reference for PCR primers: Mohamed El-Tholotha, b, Haim H Baua, and Jinzhao Songa, A Single and Two-Stage, Closed-Tube, Molecular Test for the 2019 Novel Coronavirus (COVID-19) at Home, Clinic, and Points of Entry”, Pre-Print published at doi: 10.26434/chemrxiv.11860137.v1

Forward Primer:
(SEQ ID NO: 7)
5′-Biotin-CCCTGTGGGTTTTACACTTAA-3′
Reverse Primer:
(SEQ ID NO: 8)
5′-Biotin-ACGATTGTGCATCAGCTGA-3′

The synthetic gene segment used to validate the assay is given below, that was made with the help of IDT, USA.

(SEQ ID NO: 9)
5′-CTGCTAAAGCTTACAAAGATTATCTAGCTAGTGGGGGACAACCAA
TCACTAATTGTGTTAAGATGTTGTGTACACACACTGGTACTGGTCAGG
CAATAACAGTTACACCGGAAGCCAATATGGATCAAGAATCCTTTGGTG
GTGCATCGTGTTGTCTGTACTGCCGTTGCCACATAGATCATCCAAATC
CTAAAGGATTTTGTGACTTAAAAGGTAAGTATGTACAAATACCTACAA
CTTGTGCTAATGACCCTGTGGGTTTTACACTTAAAAACACAGTCTGTA
CCGTCTGCGGTATGTGGAAAGGTTATGGCTGTAGTTGTGATCAACTCC
GCGAACCCATGCTTCAGTCAGCTGATGCACAATCGTTTTTAAACGGGT
TTGCGGTGTAAGTGCAGCCCGTCTTACACCGTGCGGCACAGGCACTAG
TACTGATGTCGTATACAGGGCTTTTGACATCTACAATGATAAAGTAGC
TGGTTTTGCTAAATTCCTAAAAACTAATTGTTGTCGCTTCCAAGAAAA
GGACGAAGATGACAATTTAATTGATTCTTACTTTGTAGTTAAGAGACA
CACTTTCTCTAACTACCAACATGAAGAAACAATTTATAATTTACTT-3′

The amplification product mixed with MRI sensitive probes are made to get maximum signal and specific to the product made during the LAMP process.

The MRI sensitive Streptavidin conjugated Super Mag nanoparticles of 50 to 150 nanometers size are used in this test depending on the magnetic field of the MRI machine used. The smaller nanobeads of 50 nm are used in large magnetic field MRIs and 100 nm large nanobeads are used in low-field MRI systems (up to 300 milli Tesla)

The mixed product undergoes a preferred process under non-uniform magnetic field or uniform magnetic field in the MRI system or both that is implemented in the preferred embodiment of magnetic separation devices used in separating the beads in other applications. In the presence of a non-uniform magnetic array of large number of magnets assembled in 96-well and 384-well format are optimized for the inventive method of detection using MRI. This process promotes the binding reaction to happen faster and this process increases the signal dramatically by magnetically enhancing the nucleic acid signal and more precisely this process increases the contrast, or strength of signal, or the signal difference between negatives and positive

The Hyperfine MRI system and Easote O-scan were used to image MRI images in 96 well plates. The Hyperfine MRI system could also measure the T2 values for the samples processed. We measured T2 values using Hyperfine MRI system and compared with bench-top NMR measurements made using 60 MHz Nanalysis Benchtop NMR Spectrometer. The measured T2 values on various NMR systems available at the university of Washington NMR Facility (Made by Bruker and Varian) to see the difference between the T2 values on various NMR between positive and negative samples. The NMR measurements and the MRI images and measurements showed that the T2 values are in the following range:

For both RT-LAMP and RT-PCR amplicons the T2 values measured between 100 milliseconds and 1800 milliseconds. The T2 values below 300 milliseconds give “darker spots” and T2 values above 500 milliseconds gives “brighter spots”. The contrast varies for values in between 300 ms and 500 ms. The brightness of the positive and negative control samples that has known virus RNA or DNA (positive) and no virus or RNA (negative), are used as the reference to decide the positives and negatives of the unknown samples on MRI using the software that helps with the data processing. The image processing software is used to make the decision on MRI data, that is developed using MATLAB and Fiji (ImageJ), that allows automated processing of the MRI images collected from samples.

This example shows how NMR also can be used along with to identify the same amplicons and the nanoparticles used are the same for both NMR and MRI. This invention can be implemented using NMR in situations there are small number of samples available.

The NMR gives the value of T2 when measurements are performed using custom pulse sequences provided by the manufacturer (Nanalysis). These pulse sequences are named differently in each machine and in Nanalysis we used One the method used to determine the T2 is the CPMG (Carr-Purcell-Meiboom-Gill) experiment because in this sequence one can refocus the field inhomogeneity effect incase of magnetic field inhomogeneities are present in the system.

The T2 values not only gives qualitative information but also give quantitative information to some extent depending on the RT-LAMP and RT-PCR amplification time and viral loads in the samples. The present invention is also used to quantify the RT-PCR and RT-LAMP product at shorter amplification times between 10 and 90 minutes of amplification to quantify various amounts of viral load or DNA.

The MRI image is collected using a clinical MRI device using the array of all the processed samples to see them visually or/and later using software program that will read, transfer and process the MRI data from 3-D into linear data showing positives and negatives very fast. The MRI images are processed using available image processing software tools such as Fiji (ImageJ) and Matlab programs developed for this purpose to declare the results of the test automatically using the software that can be used with all the MRI machines available for measuring signal from the reaction tubes.

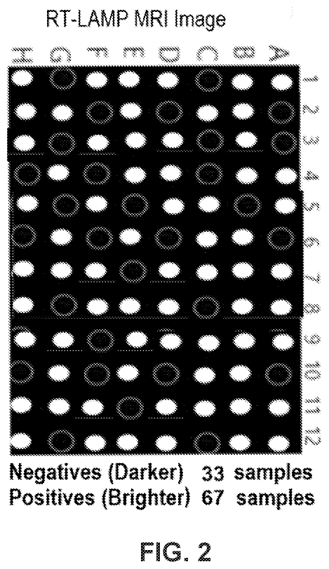

The drawings in FIGS. 2a and 2b show the MRI images obtained from RT-LAMP and the FIG. 2c show the MRI image obtained from RT-PCR. There is no qualitative difference between MRI images obtained from RT-LAMP and RT-PCR.

### DESCRIPTION OF DRAWINGS

The accompanying drawings, which are incorporated in and constitute a part of the specification, illustrate an embodiment of the invention, and together with the description, serve to explain the principles of the invention.

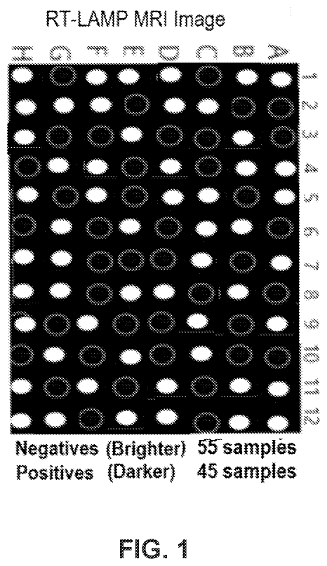

FIG. 1: The representative MRI image obtained from LAMP reaction for positive and negative samples are shown in an embodiment of the invention. This invention requires the Biotin functional group attached at 5′ end of the primer with the list of LAMP primers and their respective locations identified above. The image in which darker images represent positives and brighter imager represent negative assays. This embodiment also associated with the size of the nanoprobe bound to the product during the end-to-end assay process. The darker positives and bright negatives is achieved by specific size primers below 100 nm size (Examples: 10 nm, 20 nm, 30 nm, 40 nm, 50 nm, 60 nm, 70 nm, 100 nm and the values in between). Though the same can be extended to other sizes the best contrast was achieved between the sizes and ideally close to 50 nm.

The FIG. 1 with darker or black positives and lighter or brighter negatives show visually to the naked eye as well as measured and processed using software. These intensities vary and the difference between positive and negative can be enhanced using different MRI pulse sequences, pulse parameters and these parameters are adjusted based on the measured or known relaxation parameters such as T1 and T2 and other relaxation parameters associated with them. The MRI pulse sequence and magnetic field pulse gradients play important role in adjusting the relative brightness or intensities and sizes of dark and bright spots. So, the MRI image contrast is decided by these magnetic resonance relaxation parameters in the presence MRI static, pulsed magnetic fields and the field gradients.

FIG. 2: The representative MRI image obtained from LAMP reaction for positive and negative samples are shown in another second embodiment of the invention. This invention requires the Biotin functional group attached at 5′ end of the primer with the list of LAMP primers and their respective locations identified above. The image in which darker images represent negatives and brighter imager represent positives assays. This embodiment also associated with the larger size of the nanoprobe bound to the product during the end-to-end assay process. The brighter positives and darker negatives is achieved by specific size primers between 50 and 400 ideally around 100 nm size (Examples: 50 nm, 75 nm, 100 nm, 125 nm, 150 nm, 200 nm, 250 nm, 300 nm, 400 nm and the values in between). Though the same can be extended to other sizes the best contrast was achieved between the sizes and ideally around 50-150 nm range.

The FIG. 2 showing bright positives and darker negatives also require further reagents that are used to enhance the binding that leads to bright positives while the assay is processed using specific reagents that can alter or switch the binding process between negatives and positives. These special reagents acts on the Biotin attached to the primers and streptavidin attached to the magnetic beads and their relative interactions and binding in the presence of the magnetic fields. The “Nano switching” between the positives and negatives are happening due to three elements that has to work together as a system in giving different relation times and finally different brightness/contrast for the positives. The reason for this switching between FIG. 1 described embodiment and FIG. 2 described embodiment happens due to multiple forces acting and their relative strengths, multiple sizes of nanobeads, primers, LAMP products. This is further modified by the presence of buffers and reagents that has pH values, viscosity and intrusiveness, and the interplay between above parameters and properties. The second embodiment has been successful due to the optimization of above parameters in a systematic manner in a multi-dimensional experimental approach. The use of LAMP and Sample processing buffers also interferes with these processes, all that has been optimized, validated and finalized in this embodiment and the consistency of the results has been demonstrated under this complex parameters space.

According to an exemplary embodiment of the invention, FIG. 1 and FIG. 2 illustrates the 3-dimensional image collected using MRI machine representing each assay processed from individual sample identified by one black or white spots that are represented by circular disk. As per this preferred embodiment of the MRI detection the goal is to develop a high throughput detection modality capable of simultaneously screening hundreds or even thousands of assays for viruses and bacteria. Such a modality can be used for applications such as large population screening, rapid diagnosis during a pandemic outbreak, and real-time tracking of the spread of disease during such an outbreak. High throughput detection can also help reduce diagnosis times in general. The method proposed in this study is based on a novel technique that measures changes to the nuclear magnetic resonance relaxation time T₂ in the assay to determine the presence of a given target pathogen. It is proposed here to use commercial magnetic resonance imaging (MRI) systems available in hospitals, to read multiple assays simultaneously using T₂ weighted images that will discriminate between those that have the target virus such as Covid-19 Coronavirus and those that do not. This MRI-based method can leverage the existing installed base of MRI scanners, including fixed systems in hospitals and MRI centres, mobile trailer mounted systems, and smaller, less expensive, portable systems enabling wide patient access.

The preferred embodiment of the invention allows the average MRI system to detect up to 4000 assays every 5 to 15 minutes, including the loading and unloading time. The pre-processing of the number of samples into detectable assay has no limitations on how many samples can be processed in the same time. The LAMP or RT-LAMP assay can be processed in 20 to 30 minutes (excluding pre and post processing steps such as sample lysis and magnetic processing, with the MRI detection taking relatively shorter time, making it faster than most polymerase chain reaction (PCR) assays when calculated per sample.

According to an preferred embodiment of the invention the Magnetic Resonance Imaging (MRI) involves obtaining images with spatially resolved signals from a sample using an appropriate combination of magnetic field gradients and RF pulses. In two-dimensional imaging, a magnetic gradient is applied, say in the Z direction to select a slice of the sample in the XY plane for the measurement.

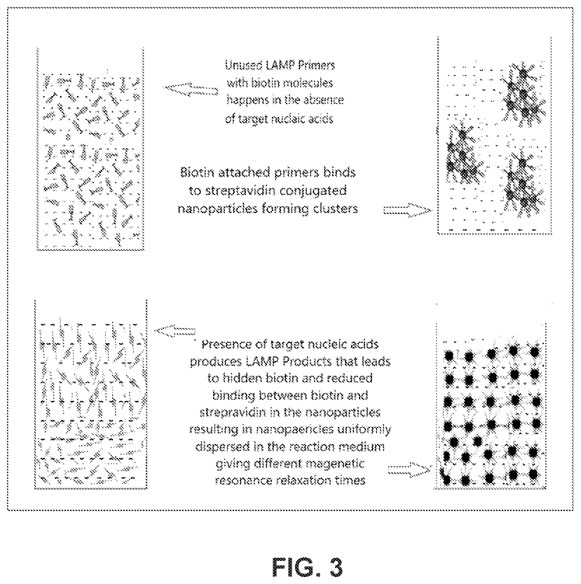

According to an preferred embodiment of the invention, FIG. 3 illustrates the major steps for the MRI detected RT-LAMP amplification assays.

According to an exemplary embodiment of the invention, the description and figures shown below illustrates major steps of the MRI detected RT-LAMP amplification assay. These steps are similar to the optically detected RT-LAMP and applied to pathogens detection. Though MRI detected assays are similar to optically detected RT-LAMP assays at the same time they are significantly different due to the fact that the nanoparticles are used as probes that are MRI sensitive. The binding chemistry of the MRI sensitive nanoparticles with the primers and assay products are significantly different. The steps are described with the representative figures to show the changes expected on the RNA and cDNA sequences synthesized prior to the MRI detection of target pathogen.

After the LAMP or RT-LAMP reaction, the magnetic or super mag nanoparticles with the surfaces are modified with streptavidin to bind to biotin molecules that are present in the reaction medium. The binding is strong for to the LAMP primers especially when the two primers FIP and/or BIP are biotinylated (biotin attached at the 5′-end). Various permutations as the sites where biotin can be added to the primers were listed above, and all the combinations are validated and the results demonstrated the inventive approaches shown in the FIG. 1 and FIG. 2.

The FIG. 3 shows how the magnetic nanoparticles will bind to the un-used primers after the LAMP reaction forming strong association between nanobeads, which is one of the embodiment resulting in FIG. 1 MRI image. These nanobeads in the absence of LAMP products enable the MRI detection by forming larger clusters due biotin-streptavidin binding induced aggregation in the presence of gradient magnetic fields or strong magnetic fields, resulting in long T1 and/or T2 giving bright MRI image.

The FIG. 3 shows how the nanobead hybridization happens, between segments of the LAMP amplicons that has the hidden/used primers with attached biotin molecules, is reduced by steric hindrance at the nanobead surface by the amplicons, leading to repulsion between them. Thus the nanobeads remain homogeneously distributed and dispersed in the reaction medium, leading to Short T1 and/or T2 giving dark image. In the final step, LAMP product is mixed with magnetic nanobeads solution and incubated for about 5-10 minutes in the presence of string gradient magnetic fields to enable quick binding of nanobeads with the “unused” or leftover LAMP primers.

The FIG. 3 shows how the magnetic nanoparticles will bind to the un-used primers after the LAMP reaction forming strong association between nanobeads, which is one of the embodiment resulting in FIG. 1 MRI image. These nanobeads in the absence of LAMP products enable the MRI detection by forming larger clusters due biotin-streptavidin binding induced aggregation in the presence of gradient magnetic fields or strong magnetic fields, resulting in long T1 and/or T2 giving bright MRI image.

The FIG. 3 shows how the nanobead hybridization happens, between segments of the LAMP amplicons that has the hidden/used primers with attached biotin molecules, is reduced by steric hindrance at the nanobead surface by the amplicons, leading to repulsion between them. Thus the nanobeads remain homogeneously distributed and dispersed in the reaction medium, leading to Short T1 and/or T2 giving dark image. In the final step, LAMP product is mixed with magnetic nanobeads solution and incubated for about 5-10 minutes in the presence of string gradient magnetic fields to enable quick binding of nanobeads with the Influenzas A and B have been validated with endpoint RT-LAMP assays The MRI based LAMP and RT-LAMP detection of COVID-19, Various strains of COVID-19, enabling large number of assays in minutes by stacking all the RT-LAMP assays inside the MRI coil and later processing the MRI images using automated software. The COVID and Influenza viruses with an analytical sensitivity of 20 to 100 copies RNA per reaction and a diagnostic sensitivity and specificity of 98.3% and 100% respectively was achieved by this approach.

The MRI detected PCR and RT-PCR detection was also validated for COVID-19 and shown in FIG. 4.

1. A method for detecting a target nucleic acid comprising the steps of:
a) providing a sample containing the target nucleic acid;
b) amplifying the target nucleic acid with at least one primer, wherein the at least one primer is biotinylated, thereby preparing a biotinylated target nucleic acid;
c) reacting the biotinylated target nucleic acid with streptavidin-nanoparticles, thereby forming amplified target nucleic acid-nanoparticle complexes; and
d) detecting the nucleic acid-nanoparticle complexes with MRI or NMR.

2. The method of claim 1, wherein amplifying the target nucleic acid comprises LAMP or PCR.

3. The method of claim 1, wherein amplifying the target nucleic acid comprises reverse transcription, wherein the method is optionally RT-LAMP or RT-PCR.

4. (canceled)

5. The method of claim 1, wherein the sample is a human clinical sample.

6. The method of claim 5, wherein the human clinical sample is saliva or a nasal swab.

7. The method of claim 1, wherein the sample is treated with at least one of: a chelating agent, proteinase K, guanidium hydrochloride, guanidium compositions and combinations thereof lyse or dissociate cells or release the nucleic acid from associated proteins.

8. The method of claim 7, where the method does not require isolation or purification of the nucleic acid.

9. The method of claim 1, wherein the target nucleic acid is a nucleic acid of a pathogen, optionally selected from a pathogenic virus or bacteria.

10. (canceled)

11. The method of claim 10, wherein the virus is a SARS-CoV-2 or a variant thereof.

12. A method for screening a plurality of samples for the presence of a target nucleic acid comprising the steps of:
a) providing a plurality of clinical samples containing the target nucleic acid;
b) amplifying the target nucleic acid in each of the plurality of clinical samples with at least one primer, wherein the at least one primer is biotinylated, thereby preparing a plurality of biotinylated target nucleic acids samples;
c) reacting each of the plurality of biotinylated target nucleic acid samples with streptavidin-conjugated nanoparticles, thereby forming a plurality of amplified target nucleic acid-nanoparticle complexes; and
d) detecting the plurality of nucleic acid-nanoparticle complexes with MRI or NMR.

13. The method of claim 12, wherein amplifying the target nucleic acid comprises LAMP or PCR.

14. The method of claim 12, wherein amplifying the target nucleic acid comprises reverse transcription.

15. The method of claim 12, wherein amplifying the target nucleic acid is RT-LAMP or RT-PCR.

16. The method of claim 12, wherein the plurality of samples are human clinical samples, wherein optionally the human clinical samples are saliva or nasal swabs.

17. (canceled)

18. The method of claim 12, wherein the plurality of clinical samples are treated with at least one of: a chelating agent, proteinase K, guanidium hydrochloride, guanidium compositions and combinations thereof lyse or dissociate cells or release the nucleic acid from associated proteins.

19. The method of claim 18, where the method does not require isolation or purification of the nucleic acid.

20. The method of claim 12, wherein the target nucleic acid is a nucleic acid of a pathogen.

21. The method of claim 20, wherein the pathogen is a pathogenic virus or bacteria.

22. The method of claim 21, wherein the virus is a SARS-CoV-2 or a variant thereof.

23. The method of claim 12, where the MRI 3-D MRI, which simultaneously detects the plurality of nucleic acid-nanoparticle complexes.


SEQUENCE LISTING (10 sequences)

SEQ ID NO: 1 - DNA, 18 bp, Artificial Sequence
  Synthetic primer
cggtggacaaattgtcac

SEQ ID NO: 2 - DNA, 22 bp, Artificial Sequence
  Synthetic primer
cttctctggatttaacacactt

SEQ ID NO: 3 - DNA, 51 bp, Artificial Sequence
  Synthetic primer
tcagcacacaaagccaaaaatttatttttctgtgcaaaggaaattaaggag

SEQ ID NO: 4 - DNA, 49 bp, Artificial Sequence
  Synthetic primer
tattggtggagctaaacttaaagccttttctgtacaatccctttgagtg

SEQ ID NO: 5 - DNA, 28 bp, Artificial Sequence
  Synthetic primer
ttacaagcttaaagaatgtctgaacact

SEQ ID NO: 6 - DNA, 27 bp, Artificial Sequence
  Synthetic primer
ttgaatttaggtgaaacatttgtcacg

SEQ ID NO: 7 - DNA, 21 bp, Artificial Sequence
  Synthetic primer
ccctgtgggttttacacttaa

SEQ ID NO: 8 - DNA, 19 bp, Artificial Sequence
  Synthetic primer
acgattgtgcatcagctga

SEQ ID NO: 9 - DNA, 619 bp, Artificial Sequence
  Synthetic copy of ORFab of SARS-CoV-2
ctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttactt

SEQ ID NO: 10 - DNA, 480 bp, Coronavirus SARS CoV-2
gctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtga